(19) United States
(12) Patent Application Publication
(10) Pub. No.: US 2025/0161446 A1
(43) Pub. Date: May 22, 2025

(54) MODIFIED IMMUNE CELLS FOR TREATING AND/OR PREVENTING METASTASIS

(71) Applicant: TECHNISCHE UNIVERSITÄT MÜNCHEN, MÜNCHEN (DE)

(72) Inventors: Percy KNOLLE, MÜNCHEN (DE); Anna-Marie PEDDE, MÜNCHEN (DE); Sebastian LACHER, MÜNCHEN (DE); Jan BÖTTCHER, MÜNCHEN (DE)

(21) Appl. No.: 18/837,588

(22) PCT Filed: Feb. 28, 2023
(86) PCT No.: PCT/EP2023/054896

§ 371 Date: Aug. 10, 2024

(30) Foreign Application Priority Data
Feb. 28, 2022 (EP) 22159206.6

(51) Int. Cl. A61K 40/11; A61K 40/15; A61K 40/31; A61P 35/04; C12N 5/0783; C12N 9/22; C12N 15/113

(52) U.S. Cl. CPC A61K 40/11; A61K 40/15; A61K 40/31; A61P 35/04; C12N 5/0636; C12N 5/0646; C12N 9/22; C12N 15/113; C12N 2310/20; C12N 2506/03; C12N 2506/11; C12N 2506/30; C12N 2510/00

(57) ABSTRACT

The present invention relates to modified immune cells, in particular modified NK cells and T cells and tumor-infiltrating lymphocytes (TILs). The present invention relates to the modified NK cells and T cells and TILs for use in the treatment and/or prevention of cancer, in particular in the treatment and/or prevention of metastases. The present invention further relates to a method of generating modified NK cells or T cells or TILs. The present invention further relates to compositions comprising said modified immune cells.

### BACKGROUND OF THE INVENTION

The present invention relates to modified immune cells, in particular modified NK cells and T cells and tumor-infiltrating lymphocytes (TILs). The present invention relates to the modified NK cells and T cells and TILs for use in the treatment and/or prevention of cancer, in particular in the treatment and/or prevention of metastases. The present invention further relates to a method of generating modified NK cells or T cells or TILs. The present invention further relates to compositions comprising said modified immune cells.

Our immune system is uniquely able to detect the neoplastic transformation of host cells, i.e. cancer. Cancer immunosurveillance critically relies on the activity of so-called natural killer (NK) cells, which express a repertoire of immune receptors enabling them to recognize and eliminate cancer cells. Multiple studies have demonstrated that NK cells fulfill a central role in the elimination of cancer metastasis (Glasner et al., Immunity, 2018; Morvan and Lanier, Nature Reviews in Cancer, 2016), which is in part mediated by their ability to produce immunostimulatory cytokines and chemokines, such as IFNγ, TNF-alpha, XCL1 and CCL5.

Up to date, various strategies have been designed to exploit the potential of NK cells for treatment of metastatic disease (Bald et al., Nature Immunology, 2020; Lorenzo-Herrero et al., Cancers, 2019; Guillerey et al., Nature Immunology, 2016). Such strategies aim to enhance immunosurveillance of metastases by boosting NK cell proliferation and persistence or by stimulating activity and effector function of NK cells in vivo. However, metastatic cancer cells can develop multiple suppressive mechanisms that enable evasion of NK cell surveillance. Such suppressive mechanisms are currently thought to be the major reason why NK cell-based immunotherapies for treatment of metastasis show only limited success in the clinic. These strategies include the downregulation of molecules that confer tumor recognition and activation of NK cells, or the production of immunomodulatory molecules that directly or indirectly suppress NK cell functions.

One suppressive mediator that has been suggested to limit anti-metastatic immunity is the eicosanoid prostaglandin E₂ (PGE₂) (Morvan and Lanier, Nature Reviews in Cancer, 2016; Wang and DuBois, Trends Mol Med, 2016; Zelenay et al., Cell, 2015; Böttcher et al., Cell, 2018). Increased PGE₂ levels resulting from enzymatic activity of cyclooxygenases (COX) 1 and 2 within primary tumors and metastatic tissue have been found in various human malignancies including colon, breast and pancreatic cancer, and high PGE₂ levels are associated with poor prognosis of cancer patients with metastatic disease. Therapeutic strategies designed to block production of PGE₂ by the use of inhibitors such as steroids (inhibitors of Arachidonic acid release) and nonsteroid anti-inflammatory drugs (blockers of cyclooxygenases) have proven ineffective in limiting the level of PGE₂ in tumor or metastasis tissue and failed to impact on patient survival (Burn et al., Lancet, 2020; Guo et al., Medicine, 2019), and by design cannot selectivity target inhibitory PGE₂ signaling on key immune cells.

Recent studies indicated that PGE₂ can directly signal onto NK cells to suppress their activity within primary tumors, identifying these cells as a key cellular target of PGE₂ signaling in solid tumors (Böttcher et al., Cell 2018, Bonavita et al., Immunity 2020). However, the mechanisms how PGE₂ signaling regulates NK cell biology and function and its consequences for NK cell-mediated control of metastasis in vivo have hardly been investigated so far. It is unknown whether certain receptors expressed on NK cells can be blocked to disable PGE₂-mediated suppression and thereby enable NK cells to eliminate metastases. Similarly, it is unclear whether blocking PGE₂ signaling on TILs such as CD8⁺ T cells can be used as a strategy to achieve tumor elimination in vivo.

There is a need in the art for improved methods and means for the treatment and/or prevention of cancer and especially metastases.

### SUMMARY OF THE INVENTION

According to the present invention this object is solved by a modified natural killer (NK) cell or a modified T cell or a modified tumor-infiltrating lymphocyte (TIL), wherein the expression and/or activity of prostaglandin E receptor 2 (EP2) and of prostaglandin E receptor 4 (EP4) is selectively inhibited or eliminated.

According to the present invention this object is solved by providing the modified NK or T cell or TIL of the present invention for use in the treatment and/or prevention of metastatic disease and cancer.

According to the present invention this object is solved by a method for generating a modified NK or T cell or TIL according to the present invention, comprising the following steps:
(a) providing leukocytes, preferably lymphocytes, from a healthy subject/donor or from a patient,
(b) separating the NK or T cells or TILs and in vitro expanding the NK or T cells or TILs;
(c) genetically modifying the NK or T cells or TILs in order to selectively eliminate the expression of EP2 and EP4;
   preferably by knocking out EP2 and EP4 or by gene silencing/knockdown of EP2 and EP4 or
   modifying the NK or T cells or TILs by treating with EP2 and/or EP4 inhibitor(s),
and
(d) in vitro expanding the genetically modified NK or T cells or TILs;
(e) optional, cryopreserving the genetically modified NK or T cells or TILs obtained in step (d).

According to the present invention this object is solved by a method for the treatment and/or prevention of cancer, preferably for the treatment and/or prevention of metastasis, comprising the step of
administering to a subject in need thereof modified NK and/or T cells and/or TILs,
wherein the expression and/or activity of prostaglandin E receptor 2 (EP2) and of prostaglandin E receptor 4 (EP4) is selectively inhibited or eliminated,
preferably administering modified NK and/or T cells and/or TILs according to the present invention or modified NK and/or T cells or TILs obtained by a method of the present invention.

According to the present invention this object is solved by a composition comprising
(a) a modified NK and/or T cell and/or TIL of the present invention,
which are preferably obtained by a method of the present invention,
(b) optional, excipient(s) and/or carrier.

### DESCRIPTION OF THE PREFERRED EMBODIMENTS OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “1 to 21” should be interpreted to include not only the explicitly recited values of 1 to 21, but also include individual values and sub-ranges within the indicated range. Thus, included in this numerical range are individual values such as 1, 2, 3, 4, 5 . . . 17, 18, 19, 20, 21 and sub-ranges such as from 2 to 10, 8 to 15, etc. This same principle applies to ranges reciting only one numerical value, such as “at least 90%”. Furthermore, such an interpretation should apply regardless of the breadth of the range or the characteristics being described.

### Modified Immune Cells

As outlined above, the present invention provides a modified immune cell. In particular, the present invention provides a modified natural killer (NK) cell. In particular, the present invention provides a modified T cell. In particular, the present invention provides a modified tumor-infiltrating lymphocyte (TIL).

“Tumor-infiltrating lymphocytes” (TILs) as used herein refers to lymphocytes that have been isolated from tumor tissue or tumor-associated tissues, including the tumor stroma and the tumor itself.

In a modified NK or T cell or TIL of the present invention the expression and/or activity of prostaglandin E receptor 2 (EP2), which is encoded by the PTGER2 gene, and of prostaglandin E receptor 4 (EP4), which is encoded by the PTGER4 gene, is selectively inhibited or eliminated.

Preferably, the expression of prostaglandin E receptor 2 (EP2) and of prostaglandin E receptor 4 (EP4) is selectively inhibited or eliminated by genetic modification.

In one embodiment, the prostaglandin E receptor 2 (EP2) and the prostaglandin E receptor 4 (EP4) are selectively inhibited or blocked by pharmacological inhibition.

Prostaglandin E receptor 2, also known as EP2, is a prostaglandin receptor for prostaglandin E2 (PGE₂) and is encoded by the human gene PTGER2. It is one of four identified PGE₂ receptors, the others being EP1, EP3, and EP4, encoded by the human genes PTGER1, PTGER3 and PTGER4, respectively, which bind with and mediate cellular responses to PGE₂ and also, but with lesser affinity and responsiveness, certain other prostanoids.

The EP2 and EP4 prostanoid receptors are two of the four subtypes of receptors for prostaglandin E₂ (PGE₂). They are in the family of G-protein coupled receptors and both receptors were initially characterized as coupling to Gs and increasing intracellular cAMP formation. The signalling through the two Gₛ-coupled receptors EP2 and EP4 activates the adenylate cyclase-triggered cAMP/PKA/CREB pathway, which mediates the dominant aspects of the anti-inflammatory and suppressive activity of PGE₂ in immune cells. Signaling by both receptors can further activate the β-catenin/T-cell factor (Tcf) pathway, thereby additionally regulating gene transcription. The EP2 receptor does this primarily through cAMP-dependent protein kinase (PKA), whereas the EP4 utilizes phosphatidylinositol 3-kinase (PI3K) as well as PKA. In addition, the EP4 receptor can activate the extracellular signal-regulated kinases (ERKs) 1 and 2 by way of PI3K leading to the induction of early growth response factor-1 (EGR-1), a transcription factor traditionally associated with wound healing. This induction of EGR-1 expression has significant implications concerning the potential role of PGE₂ in cancer and inflammatory disorders.

In one embodiment, the modified NK or T cell or TIL of the present invention is genetically modified by knocking out EP2 and EP4,
more preferably by using the CRISPR/Cas9 gene editing system.

In one embodiment, the modified NK or T cell or TIL of the present invention is modified or edited by knocking out EP2 and EP4,
more preferably by using zinc-finger nucleases (ZFNs), transcription activator-like effector nucleases (TALENs) and MegaTAL nucleases, or by using base editing.

In one embodiment, the modified NK or T cell or TIL of the present invention is modified by gene silencing/knockdown of EP2 and EP4,
more preferably via introduction of short-hairpin RNAs via the Sleeping Beauty transposon system, lentiviral vectors or gamma-retroviral vectors.

In a preferred embodiment, the modified NK cell of the present invention is a so-called SUPER NK cell.

Wherein SUPER refers to SUPpression by EP2/EP4 Refractory NK cells. SUPER NK cells are modified so that they show reduced expression or decreased functionality of the PGE₂ receptor EP2 and EP4, e.g. by a knockout of the EP2/EP4 encoding genes using CRISPR/Cas9 gene editing system. These SUPER NK cells are inert against their inhibition by PGE₂ in metastases and maintain their full functional potential and anti-metastatic activity following transfer.

In a preferred embodiment, the modified NK cell of the present invention is a so-called RESURRECT NK cell.

Wherein RESURRECT refers to REfractory to SUppRession by PGE₂ RECepTor signaling NK cells. RESURRECT NK cells are modified so that they show reduced expression or decreased functionality of the PGE₂ receptors EP2 and EP4, e.g. by a knockout of the EP2/EP4 encoding genes using CRISPR/Cas9 gene editing system. These RESURRECT NK cells are inert against their inhibition by PGE₂ in metastases or tumor tissue and maintain their full functional potential and anti-metastatic/anti-cancer activity following transfer.

In a preferred embodiment, the modified T cell of the present invention is a so-called RESURRECT T cell. Wherein RESURRECT refers to REfractory to SUppRession by PGE₂ RECepTor signaling T cells. RESURRECT T cells are modified so that they show reduced expression or decreased functionality of the PGE₂ receptors EP2 and EP4, e.g. by a knockout of the EP2/EP4 encoding genes using CRISPR/Cas9 gene editing system. These RESURRECT T cells are inert against their inhibition by PGE₂ in metastases or tumor tissue and maintain their full functional potential and anti-metastatic/anti-cancer activity following transfer.

In a preferred embodiment, the modified immune cells of the present invention are so-called RESURRECT immune cells. Wherein RESURRECT refers to REfractory to SUppRession by PGE₂ RECepTor signaling immune cells. RESURRECT immune cells are modified so that they show reduced expression or decreased functionality of the PGE₂ receptors EP2 and EP4, e.g. by a knockout of the EP2/EP4 encoding genes using CRISPR/Cas9 gene editing system. These RESURRECT T cells are inert against their inhibition by PGE₂ in metastases or tumor tissue and maintain their full functional potential and anti-metastatic/anti-cancer activity following transfer.

The terms “SUPER” and “RESURRECT” can be used interchangeably in this application.

In one embodiment, the modified NK or T cell or TIL of the present invention are further modified.

For example, the modified NK or T cell or TIL can be further modified, such as to enhance their function, such as by introducing component(s), e.g. a chimeric antigen receptor (CAR), a chemokine or cytokine receptor; an activating receptor; or by eliminating other genes, such as other inhibitory receptors.

In one embodiment, in the modified NK or T cell or TIL of the present invention EP2 and EP4 are selectively inhibited or blocked by pharmacological inhibition.

Examples for EP2/EP4 pharmacological inhibitors are PF-04418948 and PF-04852946 (EP2 antagonists), L-161982 (EP4 antagonist) and TPST-1459 (dual EP2 and EP4 antagonist).

### Medical Uses of the Modified Immune Cells

As outlined above, the present invention provides the modified NK or T cell or TIL of the present invention for use in the treatment and/or prevention of metastatic disease and cancer.

Preferably, the modified NK or T cell or TIL of the present invention is provided for use in the treatment and/or prevention of metastasis.

Preferably, the cancer is
a solid tumor, preferably a primary or secondary solid tumor,
   such as, but not limited to melanoma, breast cancer, colorectal cancer, pancreatic cancer;
a hematological tumor or malignancy,
   such as acute myeloid leukemia, multiple myeloma, non-Hodgkin lymphoma, Hodgkin lymphoma, B and T cell lymphoblastic leukemia;
and/or
metastases,
   preferably metastases in organs such as lung, liver, brain, bone marrow or lymphoid tissue.

Preferably, the treatment and/or prevention of cancer comprises adoptive immune cell therapy, preferably adoptive NK cell therapy, adoptive T cell therapy, CAR T cell therapy, CAR NK cell therapy and combinations thereof.

“Adoptive immune cell therapy” or “adoptive cell therapy (ACT)” refers to a form of therapy in which immune cells are transferred to tumor-bearing hosts. The immune cells have antitumor reactivity and can mediate direct or indirect antitumor effects.

In one embodiment, the treatment and/or prevention of cancer comprises an adjuvant therapy, such as after surgery of the primary tumor.

In one embodiment, the treatment and/or prevention of cancer comprises treatment of metastasis, including advanced metastasis.

In one embodiment, the treatment and/or prevention of cancer comprises a combination therapy with other immune therapies, such as checkpoint blockade inhibitors targeting PD-1, CTLA-4, and/or PD-L1.

In one embodiment, the treatment and/or prevention of cancer comprises a combination therapy with anti-programmed death (PD) protein 1 therapy.

In one embodiment, the treatment and/or prevention of cancer comprises combinations with other immune therapies and with anti-programmed death (PD) protein 1 therapy.

### Methods for Generating the Modified NK or T Cells

As outlined above, the present invention provides a method for generating a modified NK or T cell or TIL according to the present invention.

Said method comprises the following steps:
(a) providing leukocytes from a healthy subject/donor or from a patient;
(b) separating the NK or T cells or TILs and in vitro expanding the NK or T cells or TILs;
(c) genetically modifying the NK or T cells or TILs in order to selectively eliminate the expression of EP2 and EP4; or
modifying the NK or T cells or TILs by treating with EP2 and/or EP4 inhibitors, and
(d) in vitro expanding the modified NK or T cells or TILs;
(e) optional, cryopreserving the modified NK or T cells or TILs obtained in step (d).

### Step (a)

In step (a) of the method of the present invention, leukocytes are provided.

Preferably, the leukocytes are lymphocytes.

Preferably the leukocytes (preferably lymphocytes), are
from periphery blood, preferably obtained via leukapheresis,
from cord blood, such as from a cord blood bank,
from induced pluripotent stem cells, or.
from tumor tissue (TILs), preferably obtained by tumor dissociation,

In one embodiment, the leukocytes, preferably lymphocytes, are from autologous cells derived from a patient.

In one embodiment, the leukocytes, preferably lymphocytes, are allogeneic cells derived from an unrelated healthy subject/donor.

### Step (b)

In step (b) of the method of the present invention, the NK or T cells or TILs are separated from said leukocytes, preferably lymphocytes, and expanded in vitro.

Optionally, the NK or T cells or TILs can be cryopreserved before carrying out step (c).

### Step (c)

In one embodiment of step (c) of the method of the present invention, the NK or T cells or TILs are genetically modified in order to selectively eliminate the expression of EP2 and EP4.

The genetic modification is preferably by knocking out EP2 and EP4 or by gene silencing/knockdown of EP2 and EP4.

Preferably, the genetic modification is carried out by
(i) using the CRISPR/Cas9 gene editing system, or
(ii) using zinc-finger nucleases (ZFNs), transcription activator-like effector nucleases (TALENs) and MegaTAL nucleases, or
by using base editing, or
(iii) gene silencing/knockdown via introduction of short-hairpin RNAs via the Sleeping Beauty transposon system, lentiviral vectors or gamma-retroviral vectors.

In one embodiment of step (c) of the method of the present invention, the NK or T cells or TILs are modified by treating with EP2 and/or EP4 inhibitor(s).

Thereby, the NK or T cells or TILs are modified by pharmacological inhibition.

Preferably, the NK or T cells or TILs are modified in that EP2 and EP4 are selectively inhibited or blocked by treatment with EP2 and/or EP4 inhibitor(s).

Examples for EP2/EP4 pharmacological inhibitors are PF-04418948 and PF-04852946 (EP2 antagonists), L-161982 (EP4 antagonist) and TPST-1459 (dual EP2 and EP4 antagonist).

### Step (d)

In step (d) of the method of the present invention, the modified NK or T cells or TILs are expanded in vitro.

In one embodiment, the genetically modified NK or T cells or TILs are expanded in vitro.

In one embodiment, the NK or T cells or TILs which are modified by pharmacological inhibition are expanded in vitro.

### Step (e)

In step (e) of the method of the present invention, which is an optional step, the modified NK or T cells or TILs obtained in step (d) are cryopreserved.

Preferably, the genetically modified NK or T cells or TILs are cryopreserved.

Cryopreserved cells are cells that have been preserved by cooling to a sub-zero temperature (degree Celsius).

These cells may or may not be preserved in the presence of a cryoprotective agent (such as DMSO, glycerol, ethylene glycol or propylene glycol, or other) that protects cells from damage associated with storage at sub-zero temperature/freezing such as membrane damage or ice crystal formation

In one embodiment, the NK or T cells or TILs can be further modified. The method of the invention comprises the optional step of additional modification of the NK cell or T cell or TIL, such as to enhance their function.

Examples for such further or additional modification are
introducing component(s), such as a chimeric antigen receptor (CAR), a chemokine or cytokine receptor; an activating receptor;
or
the elimination of other genes, such as other inhibitory receptors.

### Methods of Treatment

As outlined above, the present invention provides a method for the treatment and/or prevention of cancer, preferably for the treatment and/or prevention of metastasis.

Said method comprises the step of
administering to a subject in need thereof modified natural killer (NK) and/or T cells and/or tumor-infiltrating lymphocytes (TILs),
wherein the expression and/or activity of prostaglandin E receptor 2 (EP2) and of prostaglandin E receptor 4 (EP4) is selectively inhibited or eliminated.

Preferably, modified NK and/or T cells and/or TILs according to the present invention or modified NK and/or T cells and/or TILs obtained by a method of the present invention are administered.

In a preferred embodiment, the method for the treatment and/or prevention of cancer comprises the following steps:
(a) providing leukocytes from a healthy subject/donor or from a patient,
(b) separating the NK or T cells or TILs and in vitro expanding the NK or T cells or TILs;
(c) genetically modifying the NK or T cells or TILs in order to selectively eliminate the expression of EP2 and EP4, or
modifying the NK or T cells or TILs by treating with EP2 and/or EP4 inhibitor(s);
(d) in vitro expanding the modified NK or T cells or TILs;
(e) optional, cryopreserving the modified NK or T cells or TILs obtained in step (d);
(f) administering the modified NK and/or T cells and/or TILs obtained in step (d) or from step (e), preferably via infusion.

### Step (a)

In step (a) of the method of the present invention, leukocytes are provided.

Preferably, the leukocytes are lymphocytes.

Preferably the leukocytes (preferably lymphocytes), are
from periphery blood, preferably obtained via leukapheresis,
from cord blood, such as from a cord blood bank,
from induced pluripotent stem cells, or.
from tumor tissue (TILs), preferably obtained by tumor dissociation.

In one embodiment, the leukocytes, preferably lymphocytes, are from autologous cells derived from a patient.

In one embodiment, the leukocytes, preferably lymphocytes, are allogeneic cells derived from an unrelated healthy subject/donor.

### Step (b)

In step (b) of the method of the present invention, the NK or T cells or TILs are separated from said leukocytes, preferably lymphocytes, and expanded in vitro.

Optionally, the NK or T cells or TILs can be cryopreserved before carrying out step (c).

### Step (c)

In one embodiment of step (c) of the method of the present invention, the NK or T cells or TILs are genetically modified in order to selectively eliminate the expression of EP2 and EP4.

The genetic modification is preferably by knocking out EP2 and EP4 or by gene silencing/knockdown of EP2 and EP4.

Preferably, the genetic modification is carried out by
(i) using the CRISPR/Cas9 gene editing system, or
(ii) using zinc-finger nucleases (ZFNs), transcription activator-like effector nucleases (TALENs) and MegaTAL nucleases, or
by using base editing, or
(iii) gene silencing/knockdown via introduction of short-hairpin RNAs via the Sleeping Beauty transposon system, lentiviral vectors or gamma-retroviral vectors.

In one embodiment of step (c) of the method of the present invention, the NK or T cells or TILs are modified by treating with EP2 and/or EP4 inhibitor(s).

Thereby, the NK or T cells or TILs are modified by pharmacological inhibition.

Preferably, the NK or T cells or TILs are modified in that EP2 and EP4 are selectively inhibited or blocked by treatment with EP2 and/or EP4 inhibitor(s).

Examples for EP2/EP4 pharmacological inhibitors are PF-04418948 and PF-04852946 (EP2 antagonists), L-161982 (EP4 antagonist) and TPST-1459 (dual EP2 and EP4 antagonist).

### Step (d)

In step (d) of the method of the present invention, the modified NK or T cells or TILs are expanded in vitro.

In one embodiment, the genetically modified NK or T cells or TILs are expanded in vitro.

In one embodiment, the NK or T cells or TILs which are modified by pharmacological inhibition are expanded in vitro.

### Step (e)

In step (e) of the method of the present invention, which is an optional step, the modified NK or T cells or TILs obtained in step (d) are cryopreserved.

Preferably, the genetically modified NK or T cells or TILs are cryopreserved.

### Step (f)

In step (f) of the method of the present invention, the modified NK and/or T cells and/or TILs obtained in step (d) or from step (e) are administered.

Preferably, the administration is via infusion or via intra-tumoral or local injection,

In one embodiment, the genetically modified NK or T cells or TILs are administered.

In one embodiment, the NK or T cells or TILs which are modified by pharmacological inhibition are administered.

### Compositions

As outlined above, the present invention provides a composition comprising
(a) a modified NK and/or T cell and/or tumor-infiltrating lymphocyte (TIL) of the present invention, and
(b) optional, excipient(s) and/or carrier.

Preferably, the modified NK or T cell or TIL according to the present invention is obtained by a method of the present invention.

Preferably, the modified NK or T cells or TILs are cryopreserved.

Cryopreserved cells are cells that have been preserved by cooling to a sub-zero temperature (degree Celsius).

These cells may or may not be preserved in the presence of a cryoprotective agent (such as DMSO, glycerol, ethylene glycol or propylene glycol, or other) that protects cells from damage associated with storage at sub-zero temperature/freezing such as membrane damage or ice crystal formation

Suitable excipient(s) and/or carrier are:
basal cell culture medium,
isotonic solution,
   such as Plasmalyte
serum albumin
proteins, nucleic acids or other components supporting the growth, survival or activation of the cells,
such as growth factors and cytokines, targeting antibodies, metabolites and nutrients.

### Preferred embodiments

### Results

1) Effect of direct PGE₂-Signaling on NK Cells and Their Immunological Function

As a first step, we characterized the PGE₂-mediated effects on NK cell function. To address this, conventional NK cells were isolated from the spleen of wildtype (WT) mice via cell sorting (for details, see Examples section) and cultured in low doses of IL2 and IL15/IL15Ra for five days. This in vitro cytokine culture allowed us to generate potent effector cells for the different assays.

NK cells are critical in anti-tumor immunity due to their ability to rapidly kill tumor cells via the secretion of cytotoxic granules. To assess whether pre-treatment with PGE₂ affects cytotoxicity of NK cells, degranulation and cytotoxicity assays were performed. Following PGE₂ treatment overnight, the ability of NK cells to degranulate upon stimulation was analyzed. Different stimulation methods were used, such as triggering of activating receptors (NK1.1 or NKp46), addition of PMA and ionomycin or co-culture with tumor cells. The release of cytotoxic granules was assessed by flow cytometric measurement of LAMP-1 staining. Stimulation of NK cells with anti-NK1.1 induced strong degranulation (indicated by high LAMP-1 staining), which was only slightly reduced by PGE₂ (FIG. 1A). Also, the granzyme B levels were unaffected when comparing the expression levels in untreated and PGE₂-treated NK cells (FIG. 1B). These results indicate that PGE₂ does not impair NK cell degranulation and thus, their ability to kill tumor cells. To confirm these findings, we directly analyzed the killing potential of NK cells by real-time impedance measurements of tumor cells upon co-culture with NK cells via XCelligence DP system (Agilent Technologies Inc., USA). Both, untreated and PGE₂-treated cells effectively eliminate the tumor cells (FIG. 1C). Besides their cytotoxicity, NK cells play an important immunoregulatory role in anti-tumor immunity. Upon stimulation, NK cells secrete an array of cytokines, chemokines and growth factors which orchestrate multi-immune responses. The impact of PGE₂ on the ability of NK cells to secrete cytokines and chemokines was analyzed following stimulation (stimulation methods as described in the Examples section) using quantitative multiplex assays and ELISA.

Anti-NK1.1 induced strong IFNγ and XCL1 release, which was markedly reduced in a dose-dependent manner in presence of PGE₂ (FIGS. 1D and E). Besides IFNγ and XCL1, PGE₂ signaling on NK cells diminished the secretion of various other effector cytokines, chemokines and growth factors upon stimulation (Figure F).

Taken together, the findings show that PGE₂ does not impair cytotoxicity but the secretion of cytokines and chemokines by NK cells. Thus, these cells were unable to use key intercellular communication pathways upon activation.

### 2) PGE₂-Signaling Stably Imprints a Dysfunctional Program in NK Cells

To determine the impact of PGE₂ signaling on the transcriptome of NK cells and to identify key regulated pathways, we performed RNA sequencing of NK cells.

For this approach, NK cells were pre-treated with PGE₂ for 1 h and then stimulated with anti-NK1.1 for further 4 h. The data were analyzed and filtered according a specific scheme (FIG. 2A). By comparing the gene expression pattern between unstimulated (untreated) and stimulated (untreated+aNK1.1), we first identified all genes that were upregulated or downregulated upon stimulation. We next analyzed the expression of these stimulation-induced genes in PGE₂-treated cells (PGE₂+aNK1.1). In the principal component analysis (PCA), we see a clear separation of untreated+aNK1.1 and PGE₂+aNK1.1 cells demonstrating that PGE₂ signaling affects the transcriptional program normally induced by anti-NK1.1 activation (FIG. 2B). We then further focused on the differences in gene expression and identified 142 stimulation-induced genes that were inversely regulated upon PGE₂ pre-treatment (the so-called Core PGE₂ genes). We then filtered for those genes whose expression was hampered due to PGE₂ treatment and performed a pathway analysis. The majority of genes belong to cytokine, chemokine and growth factor pathways (FIG. 2C). The heatmap showed exemplary effector cytokines, chemokines and growth factors that failed to be expressed upon stimulation due to PGE₂ treatment (FIG. 2D).

Next, we were interested in whether these immunosuppressive effects of PGE₂ signaling are reversible or irreversible. Therefore, we treated NK cells either constantly (48 h) or transiently (24 h then wash-off) with PGE₂ before stimulation with anti-NK1.1. Surprisingly, we still saw strong inhibitory effects on XCL1 secretion when we removed PGE₂ for 24 h (FIG. 2E). These results gave us a first hint that the transcriptional changes mediated by PGE₂ signaling are stably imprinted. To confirm this, we employed an assay for transposase-accessible chromatin (ATAC) using sequencing. This technique allowed us to identify genomic regions whose accessibility is changed upon PGE₂ treatment. For this approach, NK cells were treated as in the RNA sequencing experiment described above. First, we looked for genomic regions that normally open upon stimulation. As depicted in the volcano plot of FIG. 2F), promotor regions of effector cytokines and chemokines gained accessibility when NK cells were stimulated. In contrast, these regions failed to open in PGE₂-treated NK cells (FIG. 2G). We conclude from these results that the PGE₂-mediated NK cell dysfunctionality is indeed epigenetically imprinted.

In summary, these experiments provided a global overview about the NK cell intrinsic pathways targeted by PGE₂ signaling and showed that PGE₂ signaling on NK cells stably imprints a dysfunctional program characterized by a selective block in transcriptional regulation of effector cytokines and chemokines.

3) PGE₂-Mediated Impairment of Cytokine Production in NK Cells Depends on the PGE₂ Receptors EP₂ and EP₄

PGE₂ exerts its biological function by signaling via four different G-protein-coupled receptors (EP₁ to EP₄), which are linked to different intracellular signaling pathways. Specific inhibitors against the four PGE₂ receptors were used to determine receptor specificity of observed PGE₂ mediated effects. Blockade of EP₂ and EP₄ signaling almost reversed PGE₂ induced impairment of cytokine production (FIG. 3A). Thus, EP₂ and EP₄ mediated the immunosuppressive signaling of PGE₂ on NK cells.

We underscored our findings by the use of NK cells that are deficient in EP2 and EP4, the so-called RESURRECT NK cells. These cells were isolated from GzmB^{Cre} Ptger2^{-/-}Ptger4^{fl/fl} mice, in which only GzmB-expressing cells such as NK cells are selectively deficient in EP2 and EP4. Functional assays with the RESURRECT NK cells demonstrated that they are completely rescued from the inhibitory action of PGE₂ as they exerted strong cytokine production upon stimulation through NK1.1 (FIG. 3B).

### RESURRECT NK Cells Eliminate Lung Metastases

Deregulated metabolism of arachidonic acid is observed in many human cancers, resulting in increased PGE₂ production due to increased activity of the COX-pathway. Similarly, an increased production of PGE₂ is observed in numerous mouse cancer models, including BRAF^{V600E} cancer cells (Zelenay et al., Cell, 2015; Böttcher et al., Cell, 2018). This PGE₂-producing cancer model is therefore an ideal system in which to elucidate how PGE₂ signaling switches off NK cell immunosurveillance and to characterize its consequences for NK cell-mediated control of metastasis in vivo. To study this, we investigated the formation of lung metastasis following intravenous injection of BRAF^{V600E} tumor cells that express the fluorescent reporter mCherry, which enabled the detection of metastasis by confocal microscopy. This experimental model allowed us to track metastasis development over time, from early dissemination and seeding within the lung to metastasis outgrowth at later time points.

For the analysis by confocal microscopy, metastatic lungs were isolated and directly placed in Antigenfix (fixation solution) for 4-6 h. After washing the organs with PBS, we generated 300 μm thick vibratome sections which were stained for 2 h with Phalloidin.

In initial experiments, we compared the metastasis development in WT and GzmB^{Cre} Ptger2^{-/-}Ptger4^{fl/fl} mice 18 days after tumor cell injection. Compared to WT animals, transgenic mice with RESURRECT NK cells showed an almost complete elimination of lung metastases (FIG. 4A). Thus, RESURRECT NK cells were resistant towards the inactivation by PGE₂ and controlled the metastasis development. The process of metastasis critically depends on the ability of disseminated tumor cells to reside in tissue niches in a dormant state before their reactivation causes outgrowth of metastases. On the one hand, PGE₂ signaling on NK cells could act in the initial phase of metastasis, by preventing NK cells from eliminating disseminated tumor cells. On the other hand, PGE₂-mediated impairment of NK cell function could be critical for metastasis at later stages, for example when dormant tumor cells start to outgrow within lung tissue. To address both options, we compared the metastasis development between WT and GzmB^{Cre} Ptger2^{-/-}Ptger4^{fl/fl} mice over time. Interestingly, there was no difference in number and size of metastases between WT and the transgenic mice on day 8 (FIG. 4B, upper panel). But on day 18, there were more and larger metastases in mice with conventional NK cells, whereas mice with RESURRECT NK cells have less and only small metastases (FIG. 4B, lower panel and 4C). Thus, PGE₂ signaling on NK cells is critical for metastasis at later stages and enables the outgrowth of macrometastases.

Based on the results of our in vitro experiments, PGE₂ strongly impairs the production of anti-metastatic cytokines including IFNγ. To confirm the relevance of IFNγ production by NK cells in our metastasis model, we blocked this cytokine in WT and GzmB^{Cre} Ptger2^{-/-}Ptger4^{fl/fl} mice and analyzed the metastasis development 18 days after tumor cell injection by microscopy. Blocking IFNγ diminished metastasis control in GzmB^{Cre} Ptger2^{-/-}Ptger4^{fl/fl} mice resulting in higher number and size of metastases (FIG. 4D). In contrast, neutralization of IFNγ in WT mice had no metastasis-promoting effects compared to the WT control. Thus, IFNγ produced by RESURRECT NK cells is critical for an efficient elimination of metastases.

### RESURRECT NK Cells can be Used to Therapeutically Control Metastatic Disease

As demonstrated by the in vitro and in vivo studies, PGE₂ signaling via EP2 and EP4 shuts off NK cell communication pathways and disables immunosurveillance of metastasis. Thus, PGE₂ signaling on NK cells is an attractive target for the treatment of metastatic disease.

We thus tested the adoptive transfer of RESURRECT NK cells into mice with established metastases. For the therapeutic approach, we first isolated conventional NK cells and RESURRECT NK cells from mice and cultured the cells in low doses of IL2 and IL15/15Ra for five days. Meanwhile, we intravenously injected BRAF^{V600E} mCherry tumor cells into immunodeficient Rag2^{-/-}Il2rg^{-/-} mice. Three days after tumor cell injection, we transferred the in vitro cultured NK cells. After further 11 days, metastatic livers were isolated and analyzed. Treatment with conventional NK cells did not result in metastasis control. In contrast, the transfer of RESURRECT NK cells significantly reduced the number of liver metastases (FIGS. 5A and 5B). This demonstrates that already established metastases respond well to the therapy with RESURRECT NK cells (FIG. 5C). Thus, this therapeutic approach can be applied in more advanced diseases stages.

### PGE₂-Signaling on NK Cells Plays a Role in Human Cancer Patients

Similar to our mouse cancer model, a deregulated arachidonic acid metabolism leading to increased PGE₂-production is a hallmark of human cancers. Thus, our findings in mice are highly relevant to understand the mechanism that regulate NK cell immunity in primary tumors and metastases in human patients. To find out whether our findings in mice can be translated to the regulation of human NK cells in cancer, we isolated NK cells from human blood. These cells were then pre-treated with EP2 and EP4 inhibitors prior to incubation with PGE₂ for 16 h. Cytokine production upon stimulation with K562 was assessed by flowcytometric analysis. We observed that PGE₂ impaired the cytokine and chemokine production by human NK cells, which can be almost completely rescued by the blockade of EP2 and EP4 on NK cells (FIG. 6A-C).

To investigate the relevance of NK cell abundance and functionality in human cancers, we looked at tumor gene expression data from The Cancer Genome Atlas (TCGA) (TCGA Research Network: https://www.cancer.gov/tcga). We combined NK cell signature genes (NCR3, KLBR1, PRF1, CD160 and NCR1) with the Core PGE₂ gene set, including IFNγ, TNF, XCL1, identified in the RNA sequencing approach and correlated the gene expression with patient survival. When analyzing TCGA datasets for skin tumor and distant melanoma metastases, we observed a strong correlation between high expression of functional NK signature genes and better patient survival (FIGS. 6D and 6E). Thus, this gene signature can be used as prognostic factor for patient outcome.

In this application, the RESURRECT method is disclosed.

The resurrect method uses a unique mechanism shielding therapeutic immune cells (NK cells and/or T cells and/or tumor-infiltrating lymphocytes (TILs)) from the immunosuppressive tumor microenvironment in primary tumors and metastases by genetic deletion or pharmacological inhibition of the PGE₂-receptors EP2 and EP4.

### The RESURRECT Method is Broadly Applicable to Different Types of Cancer

RESURRECT NK Cells are Protected from Suppression by Diverse Cancer Types

To test whether the RESURRECT method is applicable to different types of cancer, we exposed conventional and RESURRECT NK cells to tumor factors produced by different cancers from a primary cancer cell library and analyzed their ability to produce the effector molecules XCL1 and IFNγ after stimulation (FIG. 7A). These analyses showed that the anti-cancer activity of conventional NK cells was strongly suppressed by the large majority of these different primary cancers (FIGS. 7B and 7C), which further correlated with the amount of PGE₂ produced by cancer cells (FIGS. 7B and 7C). In contrast, EP2/EP4-deficient RESURRECT NK cells were completely protected from suppression by tumor factors and displayed strong anti-cancer activity as measured by XCL1 and IFNγ production, even in presence of high levels of cancer-derived PGE₂ (FIGS. 7B and 7C). These results show that RESURRECT NK cells are protected from suppression by diverse types of cancer.

### RESURRECT NK Cells Efficiently Eliminate Primary Mouse Melanoma Tumors In Vivo

Next, we analyzed whether RESURRECT NK cells can also mediate elimination of primary solid tumors in vivo. We therefore transplanted BRAF^{V600E} melanoma tumors into GzmB^{Cre} Ptger2^{-/-}Ptger4^{fl/fl} mice and wildtype mice (as control) and analyzed their growth over time (FIG. 8A). BRAF^{V600E} melanoma tumors showed unabated and progressive growth in wildtype mice, demonstrating that conventional NK cells fail to eliminate primary tumors (FIG. 8B, left panel). In contrast, BRAF^{V600E} melanoma tumors were efficiently eliminated in GzmB^{Cre} Ptger2^{-/-}Ptger4^{fl/fl} mice (FIG. 8B, right panel), demonstrating that RESURRECT NK cells can eliminate primary tumors in vivo.

### RESURRECT NK Cells Efficiently Eliminate Metastasis by Diverse Cancer Types, Including Melanoma and Different Pancreatic Cancers.

Given our finding that RESURRECT NK cells can eliminate lung metastasis formed by BRAF^{V600E} melanoma cells (see FIG. 4), we investigated whether RESURRECT NK cells can also eliminate metastasis by other cancer types. To test this, we challenged wildtype and GzmB^{Cre} Ptger2^{-/-}Ptger4^{fl/fl} mice with two different types of pancreatic cancer cell lines expressing the oncogene Kras^{G12D} (FIG. 9A). Microscopy analysis of the lungs of these mice 18 days after tumor challenge showed that RESURRECT NK cells in GzmB^{Cre} Ptger2^{-/-}Ptger4^{fl/fl} mice efficiently eliminated lung metastasis for both these tumor models, whereas conventional NK cells in wildtype mice did not (FIG. 9B). These results demonstrate that RESURRECT NK cells efficiently eliminate metastasis by diverse cancer types that differ in their tissue of origin and driver oncogene.

The RESURRECT NK Cell Method Results in Increased Activity of NK Cells Towards Human Tumor Derived Cells from Cancer Patients.

Next, we tested if the RESURRECT method can result in increased activity of NK cells towards human tumor cells from cancer patients. To this end, we isolated NK cells from blood of healthy volunteers and measured their anti-cancer activity after exposure to conditioned medium (CM) of human tumor derived cells (HDTC) (FIG. 10A). CM from all human cancer samples prominently induced the expression of inhibitory transcription factor genes in NK cells in these assays and impaired the ability of NK cells to produce the effector cytokines IFNγ and TNFα (FIGS. 10B and 10C). However, this effect was lost when we used CM from HDTCs cultured in presence of celecoxib (CXB) (FIG. 10B), a potent inhibitor of the key PGE₂-producing enzyme COX-2. Taken together, these data reveal that preventing the inhibitory effect of PGE₂ on NK cells results in increased NK cell anti-cancer activity.

### Therapeutic Cytotoxic T Cells (RESURRECT T Cells) are Highly Suitable for the RESURRECT Method

Tumor-infiltrating lymphocytes with anti-cancer activity not only comprise NK cells but also cytotoxic CD8⁺ T cells. We therefore investigated whether the RESURRECT method can, similarly to NK cells, also be used to unleash the anti-cancer activity of cytotoxic CD8⁺ T cells.

Pharmacological Inhibition of EP2 and EP4 Completely Protect Mouse and Human CD8⁺ T Cells from Suppression by the Tumor-Derived Factor PGE₂ and Unleash Anti-Cancer CD8⁺ T Cell Activity.

To test if the RESURRECT method can be applied to cytotoxic CD8⁺ T cells and if this is feasible for both mouse and human cells, we isolated CD8⁺ T cells from mouse spleen or human blood, treated them with the immunosuppressive factor PGE₂ in presence or absence of inhibitors for the PGE₂ receptors EP2 and EP4, and determined their anti-cancer activity as measured by T cell expansion in response to anti-CD3/CD28+IL-12 stimulation (FIG. 11A). PGE₂ strongly impaired the ability of mouse CD8⁺ T cells to expand upon stimulation, however this was completely prevented by pharmacological blockade of the PGE₂ receptors EP2 and EP4 (FIG. 11B). Identical findings were made for human CD8⁺ T cells (FIG. 11C). These results demonstrate that tumor-derived PGE₂ impairs both mouse and human CD8⁺ T cells, and uncover that inhibitors targeting EP2 and EP4 fully protect from suppression and unleash anti-cancer T cell activity of mouse and human T cells. Therefore, the RESURRECT method can use pharmacological inhibitors for both NK cells (FIG. 3) and CD8⁺ T cells (FIG. 11).

RESURRECT CD8⁺ T Cells Generated by Genetic Deletion of EP2 and EP4 are Protected from Suppression by the Tumor-Derived Factor PGE₂.

Next, we generated CD4-Cre Ptger2−/−Ptger4fl/fl mice, in which Cre activity in CD4-expression during T cell development renders T cells selectively deficient in EP2 and EP4. When analyzed for their ability to expand following the exposure to tumor-derived factors produced from a primary cancer cell library, EP2/EP4 deficient CD8⁺ T cells were completely protected from tumor-mediated suppression and showed prominent expansion even when exposed to tumor factors containing high PGE₂ levels (FIG. 12B). Of note, this was not the case for conventional CD8⁺ T cells from wildtype mice, which were strongly suppressed in their ability to expand by tumor-derived factors (FIG. 12B). This further correlated with the levels of PGE₂ contained among tumor factors. Thus, RESURRECT CD8⁺ T cells generated by genetic deletion of EP2 and EP4 are protected from suppression by the tumor-derived factor PGE₂, and the RESURRECT method is broadly applicable to protect cytotoxic T cells from suppression by different cancer types.

### RESURRECT T Cells Efficiently Eliminate Primary Mouse Melanoma Tumors and Pancreatic Cancer

To analyze whether RESURRECT T cells can eliminate tumors formed by different cancer types in vivo, we transplanted BRAF^{V600E} melanoma cells and Panc02 pancreatic cancer cells into wildtype mice (containing only conventional T cells) or CD4-Cre Ptger2−/−Ptger4fl/fl mice (RESURRECT T cells) and analyzed tumor growth over time (FIGS. 13A and 14A). Both types of tumors showed progressive growth in wildtype mice, demonstrating that conventional T cells fail to eliminate these tumors (FIGS. 13B and 14B). In contrast, CD4-Cre Ptger2−/−Ptger4fl/fl mice efficiently rejected both BRAF^{V600E} melanoma tumors as well as Panc02 pancreatic cancer cells in all animals tested (FIGS. 13B and 14B). These data demonstrate that RESURRECT T cells can efficiently eliminate different types of primary solid tumors.

Adoptive Cell Therapy with Tumor-Specific RESURRECT T Cells Achieves Tumor Elimination

To test if RESURRECT T cells can be used for cancer therapy in a cell therapy treatment setting, we challenged wildtype mice with BRAF^{V600E} tumors expressing the model antigen Ovalbumin (OVA) followed by adoptive transfer of a small number of either OVA-specific conventional OT-I CD8⁺ T cells or OVA-specific RESURRECT OT-I CD8⁺ T cells (FIG. 15A). Analysis of tumor-infiltrating OT-I T cells demonstrated that adoptively transferred tumor antigen-specific RESURRECT T cells can give rise to large numbers of effector T cells within tumor tissue, whereas this ability is completely suppressed for conventional T cells (FIG. 15B). These results demonstrate that the RESURRECT method is applicable to tumor- infiltrating lymphocytes and can be used to unleash their anti-cancer activity at the tumor site. In addition, tumor growth analyses over time demonstrated that the adoptive transfer of RESURRECT OT-I T cells resulted in efficient tumor elimination (FIG. 15C), which was not observed for the adoptive transfer of conventional OT-I T cells that showed no benefit when compared to mice that did not receive any T cells (FIG. 15C). Therefore, treatment with RESURRECT T cells can be used for cancer treatment to achieve tumor elimination.

The following examples and drawings illustrate the present invention without, however, limiting the same thereto.

### BRIEF DESCRIPTION OF THE DRAWINGS

FIG. 1. PGE₂ impairs cytokine and chemokine production by NK cells.

(A-F) In all in vitro assays, cultured conventional NK cells were first pre-treated with PGE₂ for 16 h. (A) Release of cytotoxic granules by degranulation upon anti-NK1.1 stimulation and (B) expression of the cytotoxic effector molecule granzyme B were assessed by flowcytometric analysis. (C) NK cell mediated killing of tumor cells was measured using the XCelligence system. (D-F) Quantification of cytokines, chemokines and growth factors secreted by NK cells upon NK1.1 stimulation was done by ELISA (D-E) or multiplex cytokine assays (F).

FIG. 2. PGE₂ signaling stably imprints a dysfunctional program in NK cells.

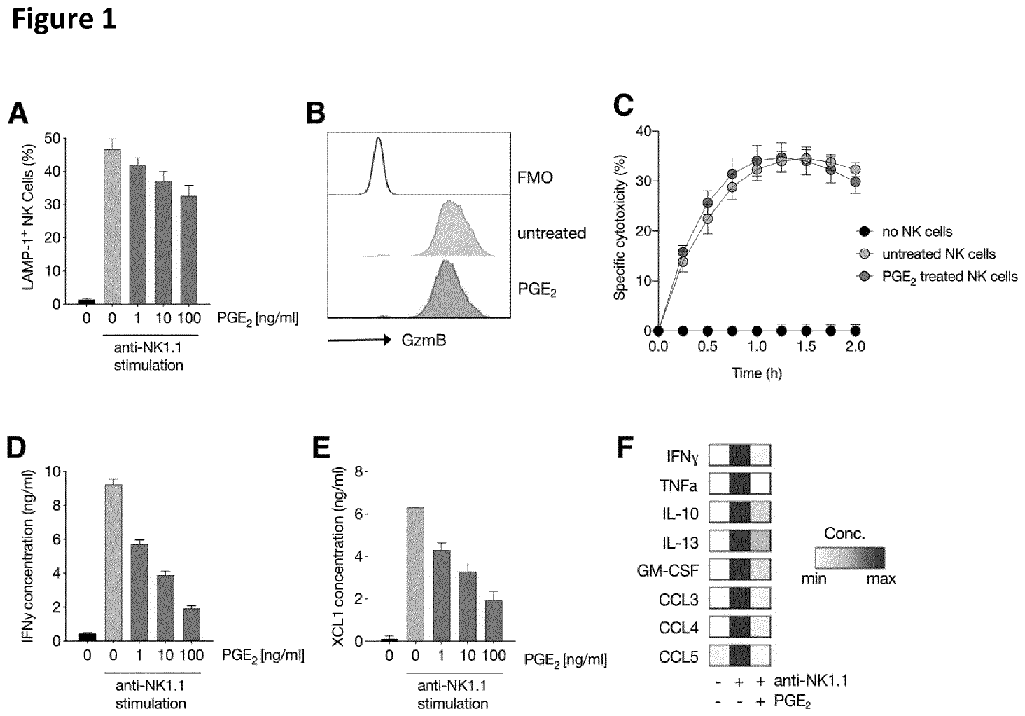

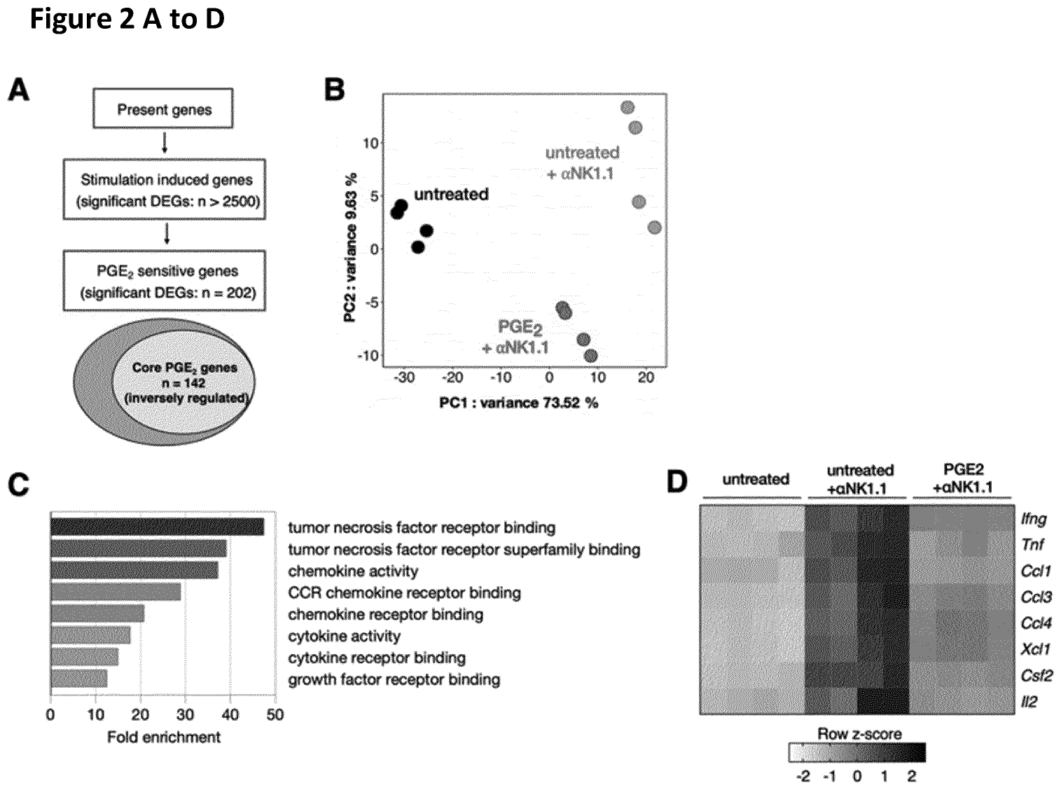

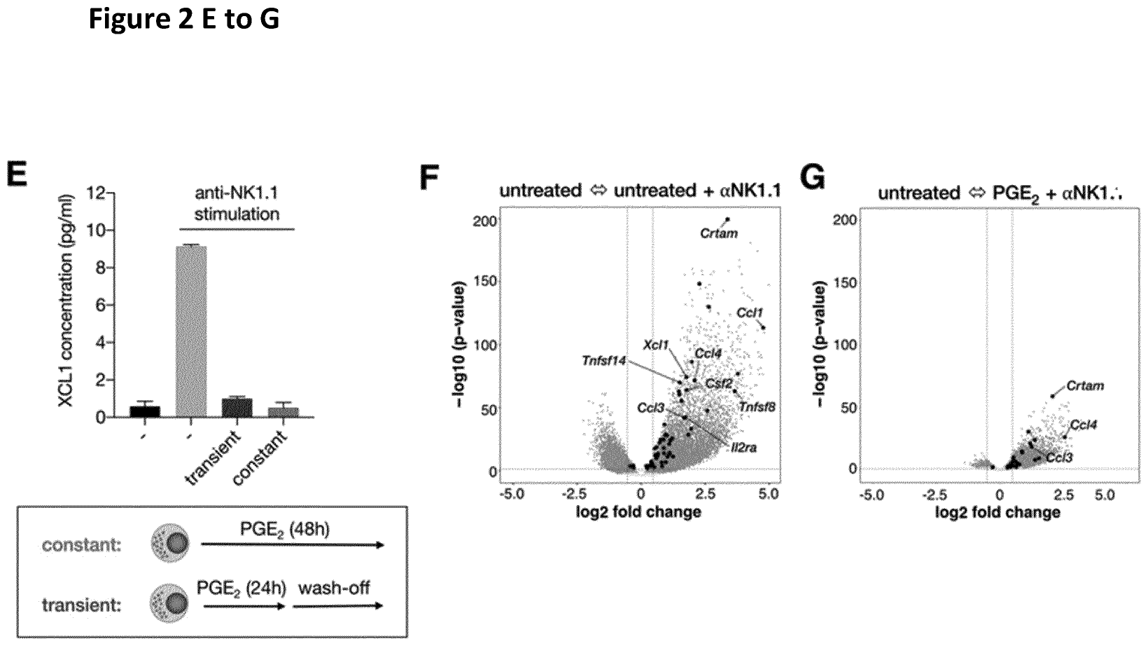

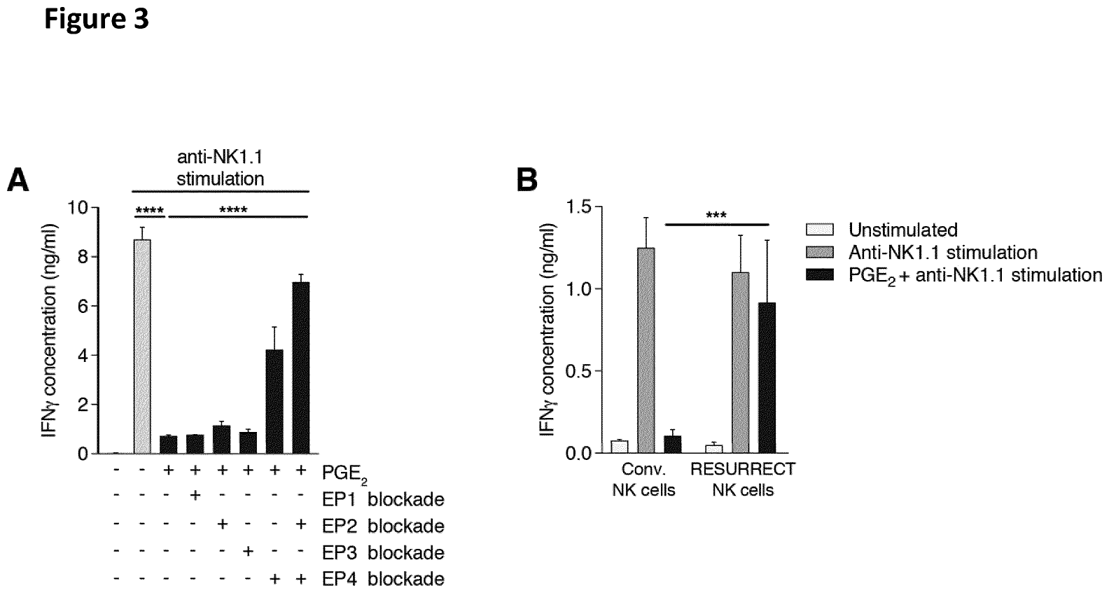

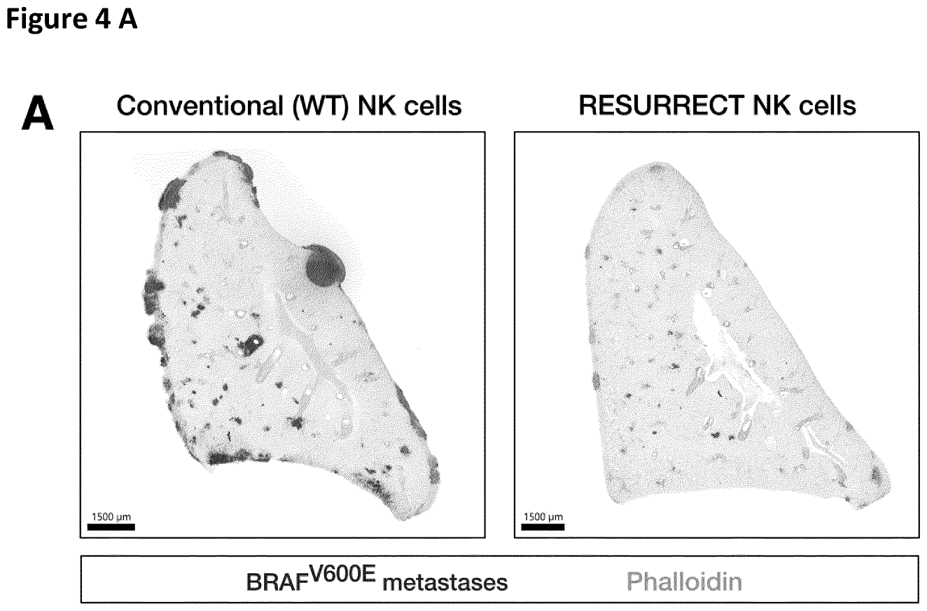

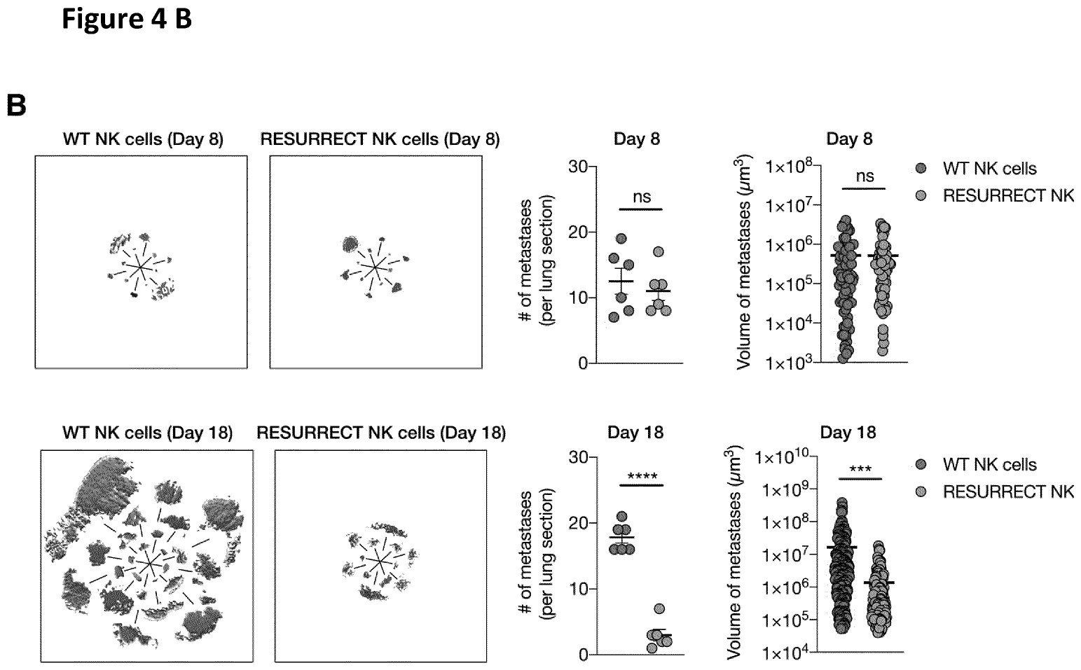

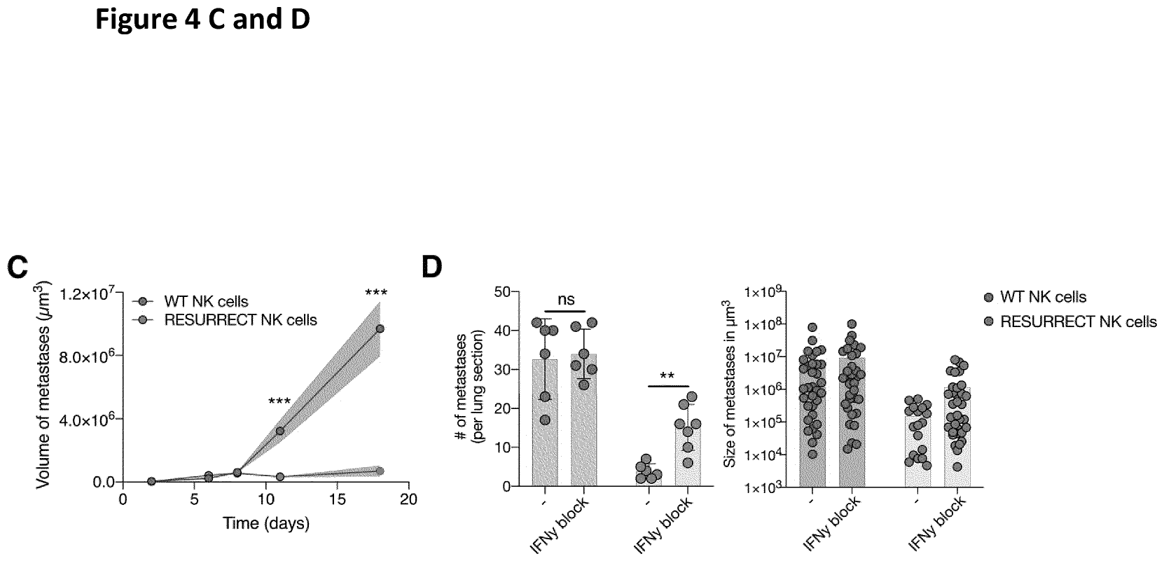

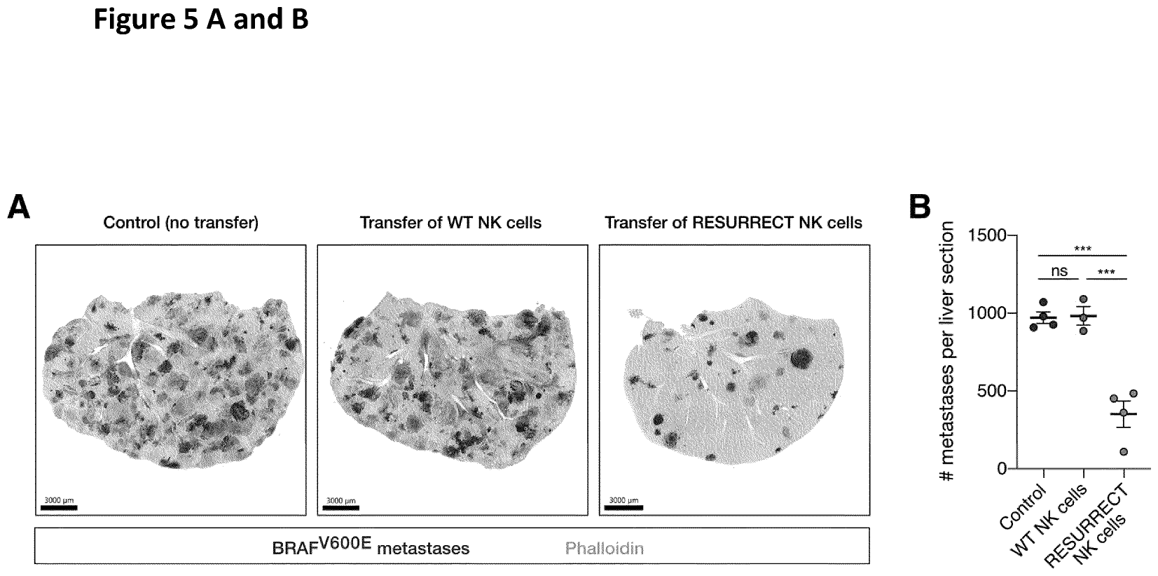

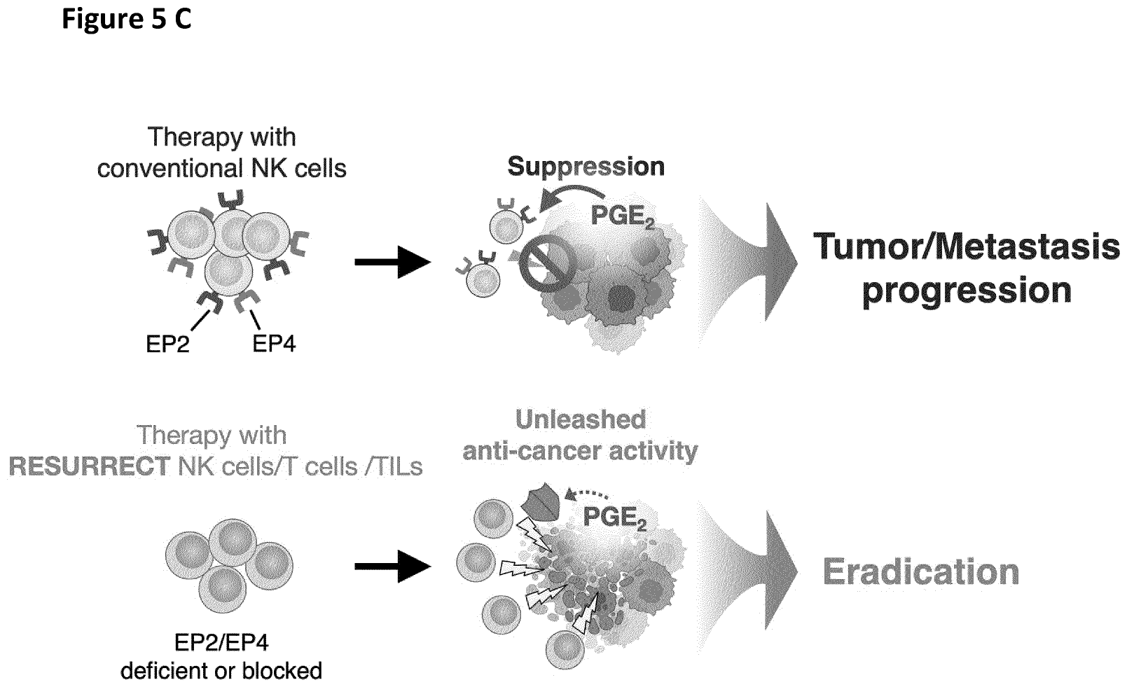

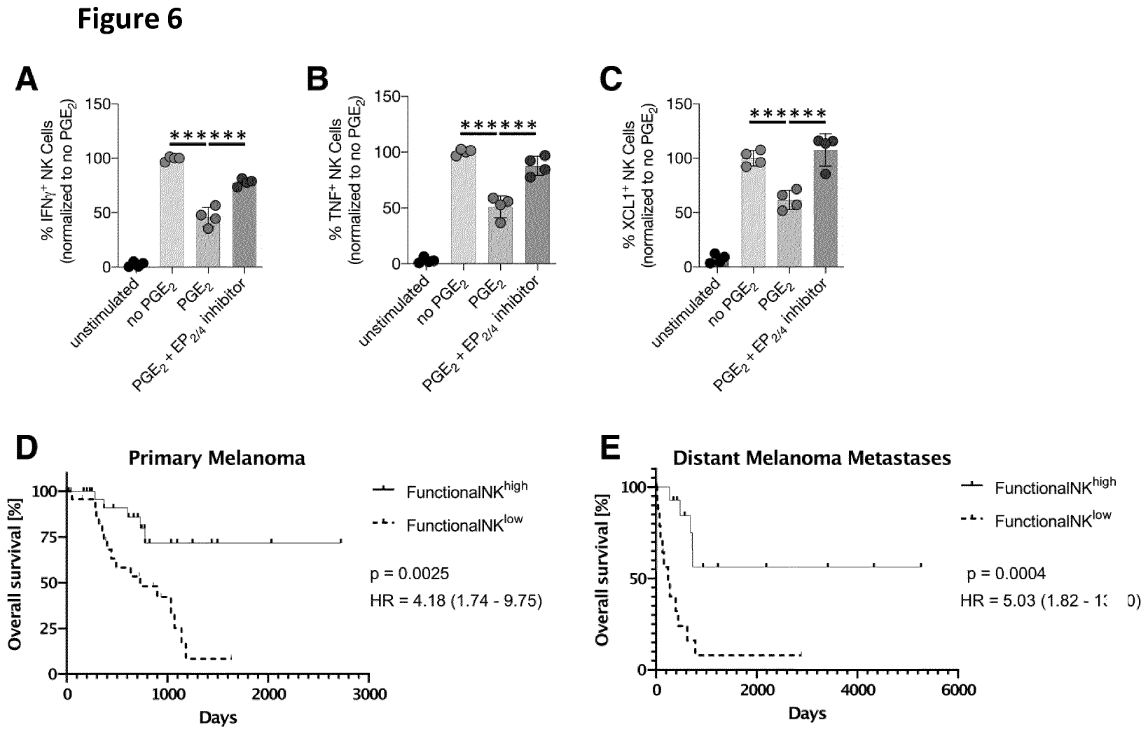

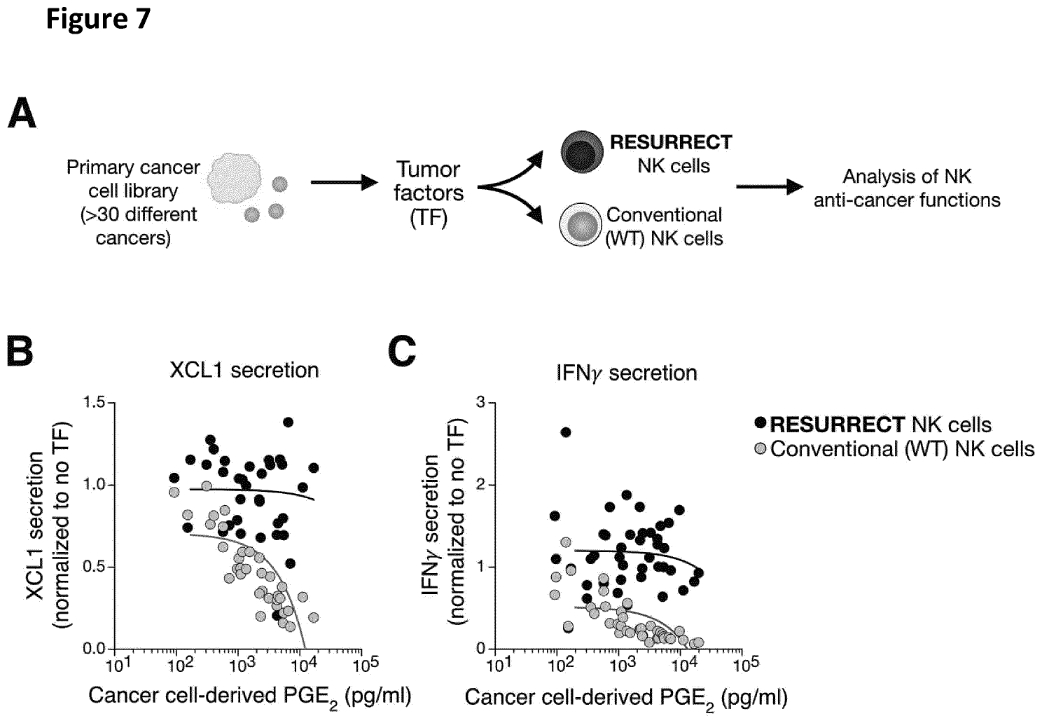

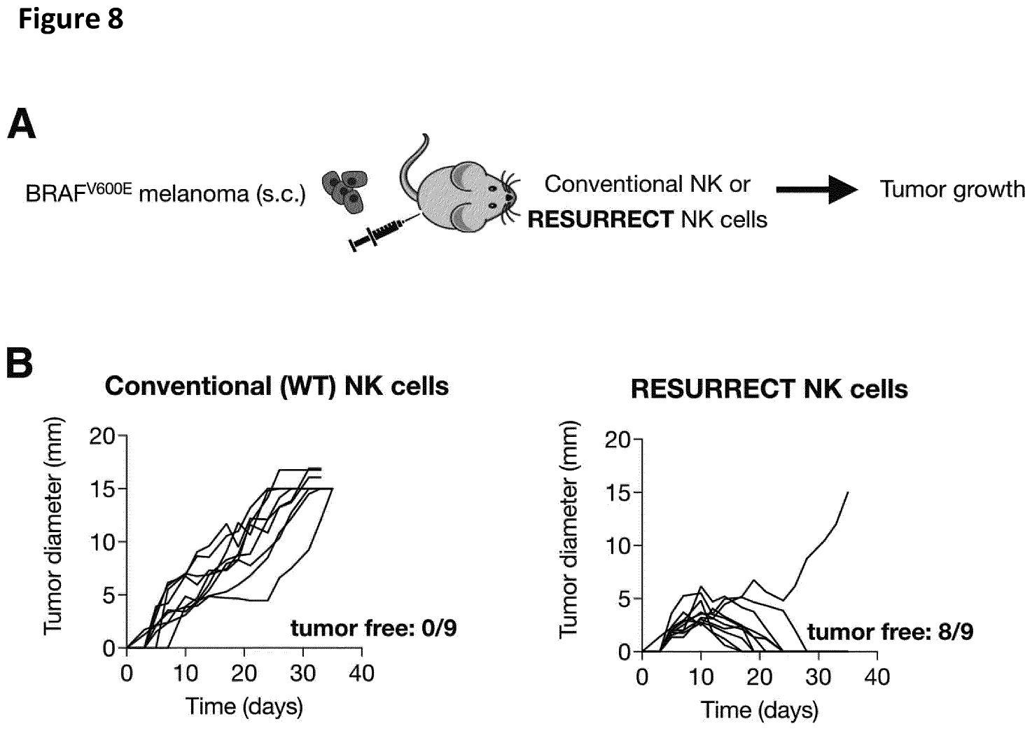

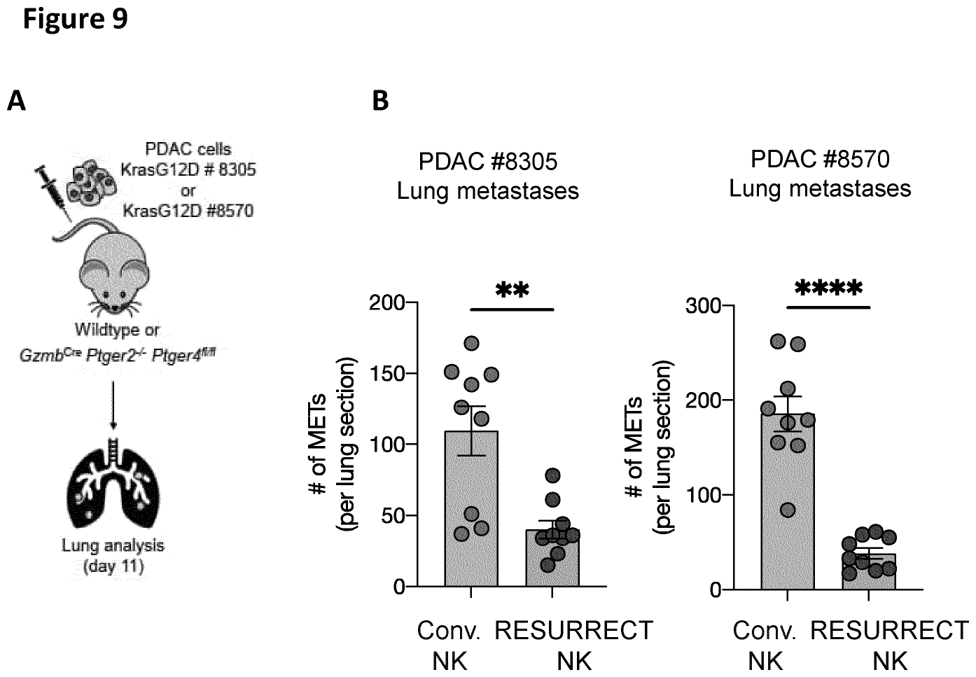

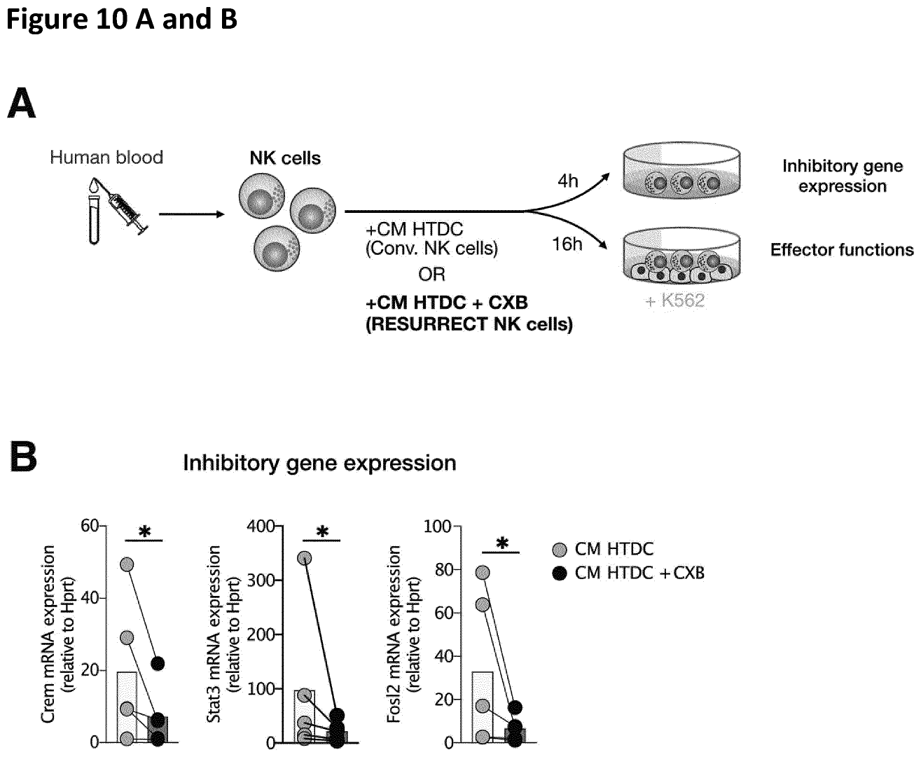

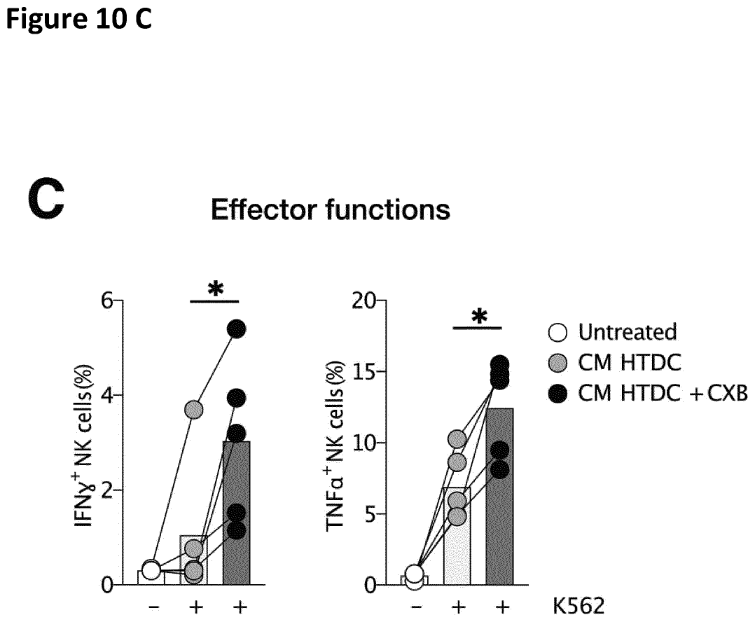

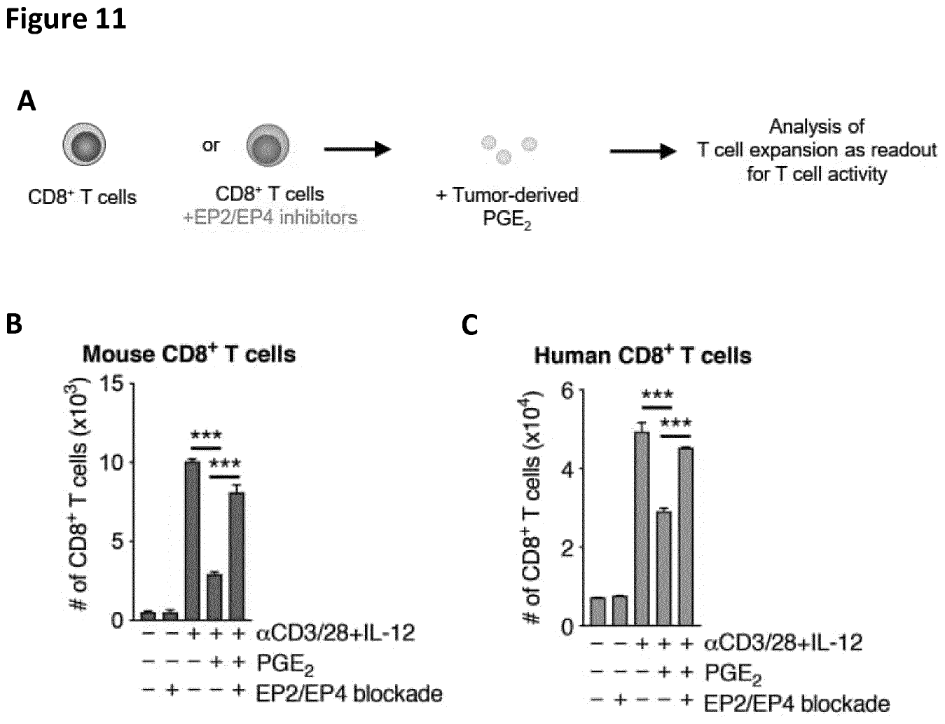

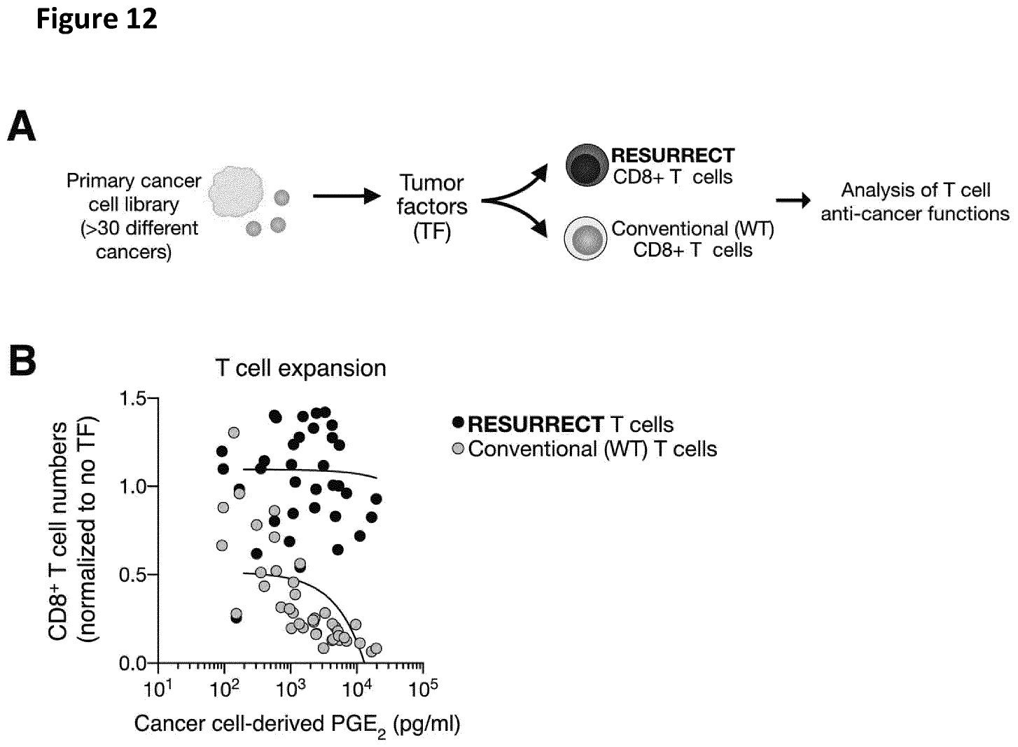

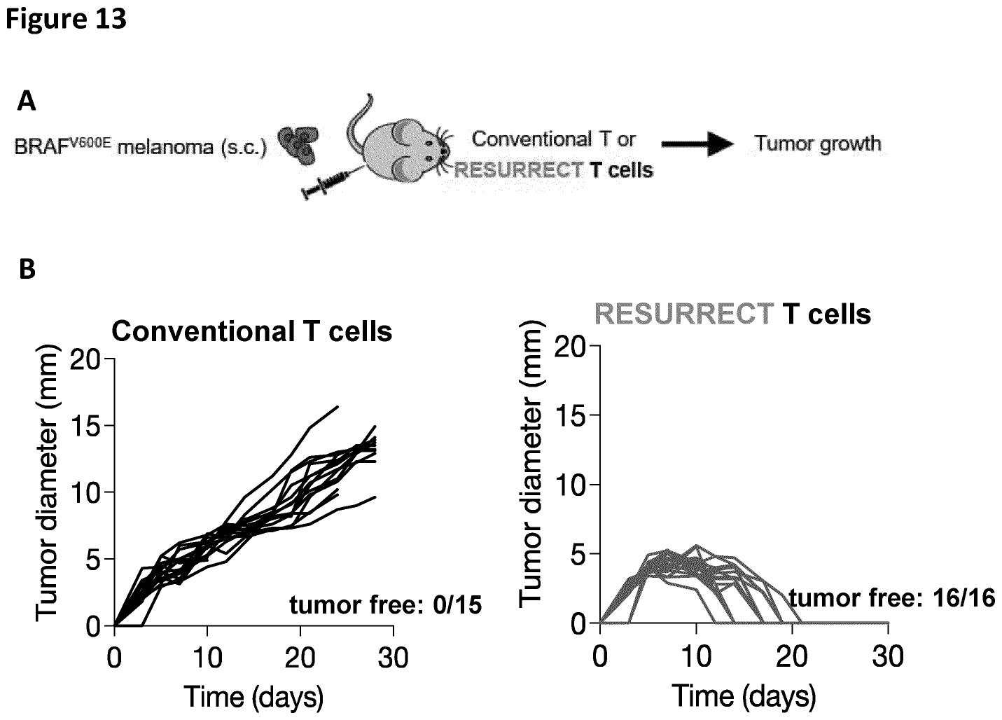

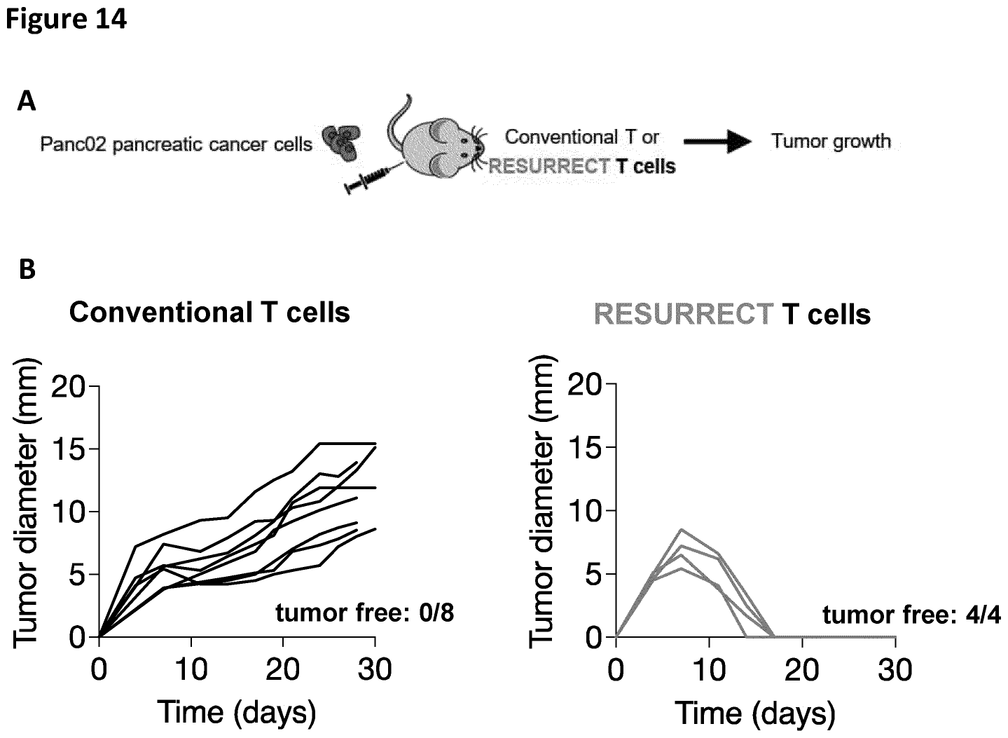

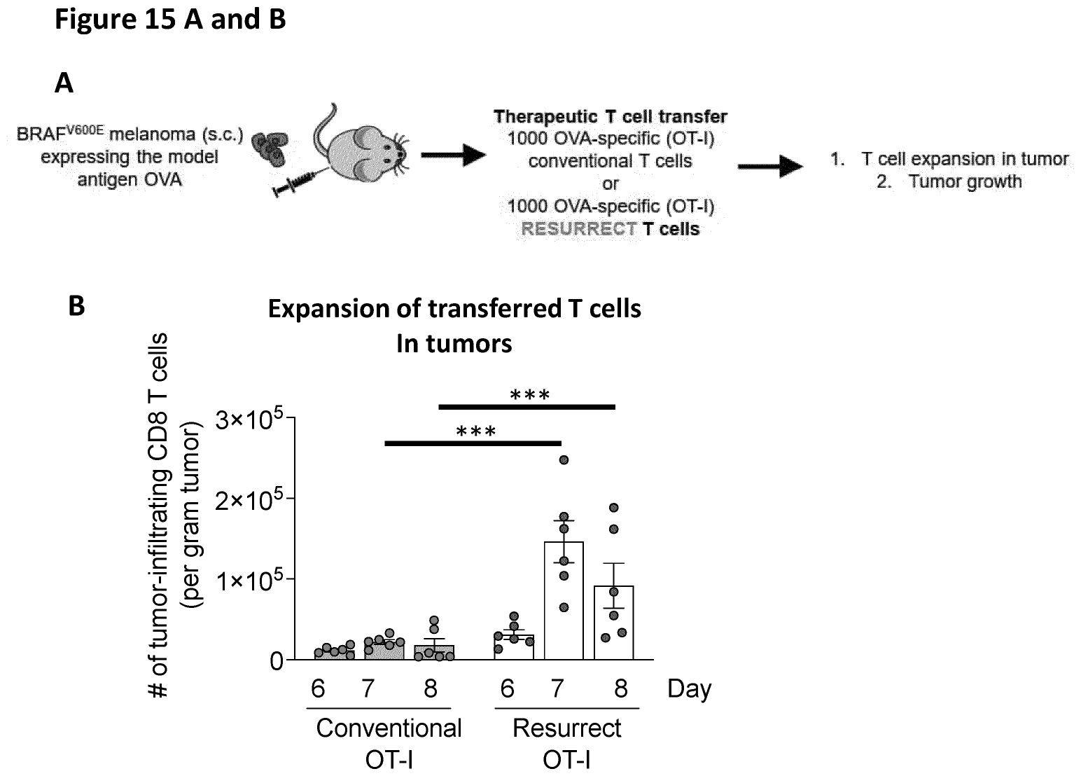

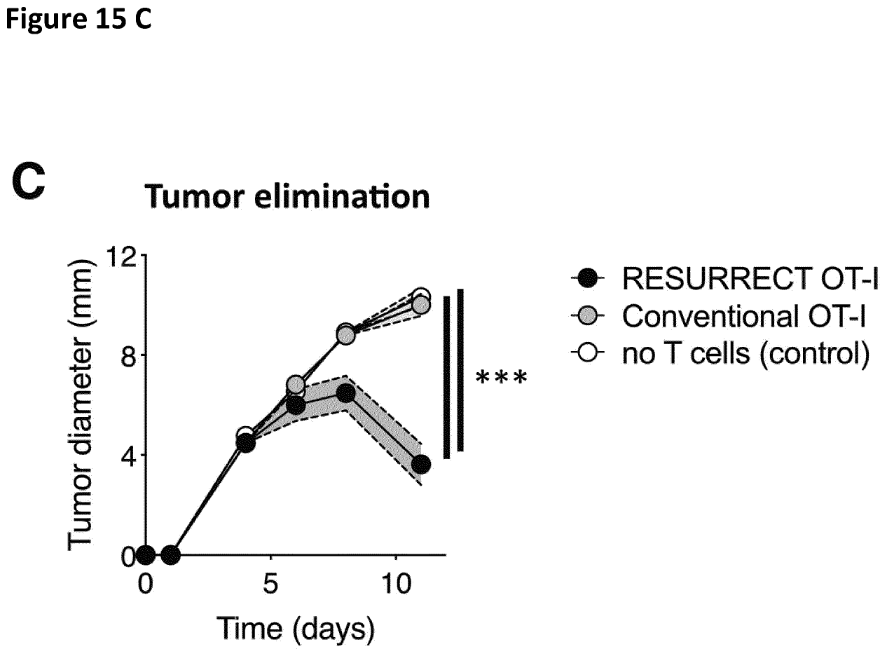

(A-D) Conventional NK cells were exposed to PGE₂ for 1 h and then stimulated by anti-NK1.1 for additional 4 h before subsequent analysis by RNA sequencing. (A) Schematic illustration of the analysis and filtering process of the sequencing data. (B) Principal component analysis (PCA) depicting differences in global gene expression for conventional NK cells (untreated), conventional NK cells stimulated with anti-NK1.1 (untreated+aNK1.1) and conventional NK cells treated with PGE2 followed by stimulation with anti-NK1.1 (PGE₂+aNK1.1). (C) Pathway analysis on core PGE₂ genes showing the eight most significantly regulated pathways blocked by PGE₂ signaling in conventional NK cells. (D) Heatmap showing protein expression of key cytokines, chemokines and growth factors involved in metastatic activity of NK cells.

(E) Effect of constant or transient treatment of conventional NK cells with PGE₂ prior to anti-NK1.1 stimulation on the production of the chemokine XCL1. XCL1 production upon stimulation was quantified in supernatants by ELISA.

(F, G) Analysis of epigenetic changes induced by PGE2 signaling in conventional NK cells by ATAC sequencing. Conventional NK cells were exposed to PGE₂ for 1 h and then stimulated for further 4 h by anti-NK1.1 prior to the sequencing. (F) Volcano plot showing differentially accessible genomic regions between conventional NK cells (untreated) and conventional NK cells activated by anti-NK1.1 (untreated+aNK1.1). Highlighted are promoter regions of genes encoding for key NK cell effector molecules that gain genomic accessibility upon stimulation.

(G) Volcano plot showing differentially accessible regions between conventional NK cells (untreated) and PGE₂-exposed conventional NK cells activated by anti-NK1.1 (untreated+aNK1.1), highlighting the inability of genomic regions to open upon NK cell stimulation following PGE₂-treatment.

FIG. 3. PGE₂ mediated impairment of cytokine production depends on EP2 and EP4.

(A) Conventional NK cells were pre-treated with synthetic inhibitors for the PGE₂-receptors EP1, EP2, EP3 and/or EP4 and subsequently incubated with PGE₂ for 16 h. IFNγ production upon anti-NK1.1 stimulation was quantified in supernatants by ELISA. (B) Conventional (WT) or RESURRECT NK cells (EP2-and EP4-deficient) were exposed to PGE₂ for 16 h and then stimulated with anti-NK1.1. IFNγ produced by NK cells was quantified by ELISA.

FIG. 4. EP2 EP4-deficient RESURRECT NK cells efficiently eliminate lung metastases.

(A-D) Development of lung metastases following intravenous (i.v.) injection of metastatic BRAFV600E tumor cells into wildtype mice (WT, harboring conventional NK cells) or transgenic mice in which GzmB-expressing cells such as NK cells are selectively deficient in EP2 and EP4 (RESURERRECT NK cells).

(A) Analysis of lung metastasis by confocal microscopy 18 days after tumor cell injection.

(B) Visualization and quantification of metastasis volume and numbers in mice with conventional (WT) and RESURRECT NK cells on day 8 (upper panel) and day 18 (lower panel) post tumor cell injection.

(C) Quantification of metastasis development over time.

(D) Impact of neutralization of the NK cell-derived cytokine IFNγ on metastasis formation. Metastases in mice with conventional (WT) or RESURRECT NK cells were analyzed 18 days after tumor cell injection.

FIG. 5. Established metastases are eliminated by RESURRECT NK cells after adoptive transfer.

(A-B) Treatment of established liver metastases in Rag2^{-/-}Il2rg^{-/-} mice (lacking endogenous NK cells) with adoptively transferred conventional (WT) or RESURRECT NK cells. Mice without NK cell transfer served as control. (A) Microscopy analysis of metastases in livers. (B) Quantification of liver metastases.

(C) Scheme showing the RESURRECT cell therapy method.

FIG. 6. PGE₂ impairs human NK cells via EP2/EP4 and is associated with poor cancer patient survival.

(A-C) Human NK cells were pre-treated with EP2 and EP4 inhibitors followed by incubation with PGE₂ for 16 h. Production of the effector molecules (A) IFNγ, (B) TNF and (C) XCL1 upon stimulation with the tumor cell line K562 was assessed by flowcytometric analysis.

(D-E) Correlation of a gene signature for functional NK cells with patient survival for (D) skin tumor samples and (E) distant melanoma metastases. The functional NK cell signature contains NK cell-specific genes and genes associated with NK cell function that have been identified to be selectively impaired by PGE₂ signaling in our core PGE₂ RNA-sequencing analysis (NCR3, KLBR1, PRF1, CD160, NCR1, IFNγ, TNF, IL2, XCL1, CCL4, CCL15, CCL3, CCL1, CSF2, IL2Ra, CRTAM, ICAM1, TNFSF9, TNFSF14, TNFSF8).

FIG. 7. RESURRECT NK cells show prominent polyfunctional anti-cancer activity against a broad range of primary cancer cells.

(A) RESURRECT or conventional NK cells (as control) were stimulated after incubation with tumor factors (TF) from a primary cancer cell library composed of multiple different pancreatic cancers).

(B) Analysis of NK cells for production of the chemokine XCL1 (a chemokine that is important to attract dendritic cells).

(C) Analysis of NK cells for production of the effector cytokine IFNγ (a cytokine for immune response activation that also has direct anti-cancer activity). Each circle represent data for a different pancreatic cancer cell line.

FIG. 8. RESURRECT NK cells efficiently eliminate primary melanoma tumors.

(A) Scheme for (B).

(B) Tumor growth following subcutaneous (s.c.) transplantation of 2×10⁵ BRAF^{V600E} melanoma into wildtype mice with conventional NK cells or GzmB^{Cre} Ptger2^{-/-}Ptger4^{fl/fl} mice with RESURRECT NK cells (n=9 per group).

FIG. 9. RESURRECT NK cells efficiently eliminate pancreatic ductal adenocarcinoma (PDAC) metastases.

(A) Scheme for microscopy-based analysis of metastasis development in the lung following injection of pancreatic ductal adenocarcinoma (PDAC) cells into the tail vein of wildtype mice (containing conventional NK cells) or GzmB^{Cre} Ptger2^{-/-}Ptger4^{fl/fl} mice (containing RESURRECT NK cells).

(B) Quantification of lung metastasis 18 days after tumor cell injection. Similar to melanoma, mice with RESURRECT NK cells efficiently eliminate metastasis formed by two aggressive KrasG12D-expressing PDAC cell lines, leading to metastasis free lungs. Each circle represents one mouse, showing robustness of the effect of RESURRECT NK cells.

FIG. 10. PGE₂ is key human tumor-derived factor suppressing the polyfunctional anti-cancer activity of human NK cells.

(A) Human NK cells were exposed to tumor factors from human tumor-derived cells (HDTC) or tumor factors from human tumor-derived cells in which production of PGE₂ was prevented by Celecoxib (CXB)-mediated inhibition of cyclooxygenase enzymes.

(B) Analysis of NK cell inhibition by measuring expression of inhibitory transcription factor genes.

(C) Analysis of NK cell anti-cancer effector function by measuring IFNγ and TNF production.

FIG. 11. Application of the RESURRECT method to mouse and human cytotoxic CD8⁺ T cells by pharmacological inhibitors of EP2 and EP4.

(A) Analysis to determine whether the method is applicable to mouse and human cytotoxic T cells by pharmacological inhibition of EP2 and EP4. Human or mouse CD8⁺ T cells were treated or not with EP2/EP4 inhibitors and exposed to tumor-derived PGE₂ for 16 h before analyses of T cell expansion 72 h later.

(B, C) Quantification of T cell expansion. While unprotected T cells are strongly suppressed by tumor factors, application of the RESURRECT method using dual blockade of EP2 and EP4 results in full protection of T cell activity, both for mouse and human cytotoxic T cells.

FIG. 12. RESURRECT cytotoxic CD8⁺ T cells are completely resistant to suppression in a broad range of primary cancer cells.

(A) RESURRECT T cells were genetically modified to be deficient in EP2 and EP4 and analyzed for their ability to resist suppression by tumor factors produced from different tumors of a primary cancer cell library (pancreatic cancer). Conventional CD8+T cells from WT mice served as control.

(B) Analysis of T cell expansion upon anti-CD3/CD28 stimulation as read out for anti-cancer T cell activity. While conventional T cells are strongly suppressed by tumor factors, RESURRECT T cells are not inhibited. Even for tumors where there is high concentration of the immunosuppressive tumor factor PGE₂.

FIG. 13. RESURRECT T cells efficiently eliminate primary melanoma tumors.

(A) Tumor growth of BRAF^{V600E} melanoma transplanted s.c. into wildtype mice with conventional cytotoxic CD8⁺ T cells or CD4Cre Ptger2^{-/-}Ptger4^{fl/fl} mice with RESURRECT cytotoxic CD8⁺ T cells.

(B) Analysis of tumor growth over time. (n=15-16 per group).

FIG. 14. RESURRECT T cells efficiently eliminate tumors formed by therapy-resistant pancreatic cancers.

(A) Tumor growth of therapy-resistant Panc02 pancreatic cancer cells transplanted s.c. into wildtype mice with conventional cytotoxic CD8⁺ T cells or CD4^{Cre} Ptger2^{-/-}Ptger4^{fl/fl} mice with RESURRECT cytotoxic CD8⁺ T cells.

(B) Analysis of tumor growth over time. (n=4-8 per group).

FIG. 15. Adoptive cell therapy with RESURRECT CD8⁺ T cells achieves potent anti-cancer T cell responses and tumor elimination.

(A) Experimental design to determine the therapeutic benefit of adoptive T cell transfer with a low number (1000 cells) of antigen-specific RESURRECT CD8⁺ T cells versus tumor model antigen-specific conventional CD8⁺ T cells.

(B) Analysis of T cell expansion within tumor tissue, showing selective expansion of RESURRECT T cells but not conventional T cells.

(C) Analysis of tumor eradication by measurement of tumor size, showing tumor elimination by adoptively transferred RESURRECT CD8⁺ T cells whereas there is no effect of conventional CD8⁺ T cells.

### EXAMPLES

### Example 1

### Materials and Methods

### Mice

All mice used in this study were bred and maintained under specific-pathogen-free conditions at the School of Medicine, Technical University Munich (TUM), according to the guidelines of the Federation of Laboratory Animal Science Association. All mice were on a C57BL/6 genetic background. The following strains were used: C57BL/6J wildtype mice (WT, as control), GzmB^{Cre} Ptger2^{-/-}Ptger4^{fl/fl} mice (in which expression of the Cre-recombinase under the GzmB promoter results in a double knockout of EP4 and EP2 selectively in GzmB-expressing NK cells), CD4^{Cre} Ptger2^{-/-}Ptger4^{fl/fl} mice (in which expression of the Cre-recombinase under the CD4 promoter during T cell development results in a double knockout of EP4 and EP2 selectively in T cells), OT-I (in which CD8⁺ T cells express a transgenic T cell receptors specific for the model antigen Ovalbumin (OVA), OT-I×CD4^{Cre} Ptger2^{-/-}Ptger4^{fl/fl}, and Rag2^{-/-}IL2rg^{-/-} (lacking NK cells and other adaptive lymphocytes). Experiments were conducted using age- and gender-matched mice in accordance with approved institutional protocols. Animals were typically 6-10 weeks old at the time of use and consisted of males and females.

### Cell Lines

BRAF^{V600E} melanoma, pancreatic ductal adenocarcinoma (PDAC) cell lines KrasG12D #8305, KrasG12D #8570, Panc02 and primary pancreatic cancer cell lines were cultured in complete RPMI medium (RPMI 1640 with 10% fetal calf serum, 50 mM 2-mercaptoethanol, 100 U/ml Penicillin, 100 mg/ml Streptomycin, 2 mM L-Glutamine). Please note that all these cell lines, similar to numerous other tumor cell models, produce large amounts of PGE₂. Cell lines expressing the mCherry reporter were generated by retroviral transduction as described previously (Böttcher et al., Cell, 2018). For generation of conditioned medium containing tumor factors (TF), tumor cells were cultured for 48 h in complete RPMI medium and the conditioned medium was collected and frozen at −20° C.

### Human Specimen

Tumor biopsies were obtained at the time of surgery and directly processed. For preparation of human tumor-derived cells (HDTC), surgical specimen were mechanically dissociated and digested with digestion buffer (DMEM mixed with 50% ready-to-use accutase solution and supplemented with 1 mg/ml collagenase IV, 1 mg/ml hyaluronidase, 10 U/ml DNase I and 10% FCS) for 60 min at 37° C. Cells were washed in PBS, filtered and frozen as single cell suspension for future use. HDTC conditioned medium was generated by culturing 2.5×10⁵ cells in 200 μl complete RPMI medium for 48 h in presence or absence of the COX-2 inhibitor celecoxib (5 μM).

### Isolation and Culture of Mouse NK Cells

For the isolation of mouse NK cells, the spleen was excised from mice and passed through a steel sieve. Red blood cells were lysed by incubating the cells with ammonium chloride potassium buffer for 2 min. Following filtration through a 70 μm cell strainer, CD49b⁺ leukocytes were purified by immunomagnetic separation to enrich for CD49b⁺ NK cells. Th cells were stained with anti-CD49b FITC, anti-CD3ε PerCP/Cy5.5, anti-CD19 PE/eF610 and CD64 PE/Cy7 for 15 min at 4° C. and NK cells, phenotypically identified as CD49b⁺CD3ε⁻CD19⁻CD64⁻ cells, were FACS-sorted using a SH800 Cell Sorter. After isolation, NK cells were cultured in low doses of IL2 and IL15/IL15Ra for five days to rest the cells and generate functional NK cells.

### Isolation and Culture of Mouse CD8⁺ T Cells

For the isolation of mouse CD8⁺ T cells, the spleen was excised from mice and passed through a steel sieve. Red blood cells were lysed by incubating the cells with ammonium chloride potassium buffer for 2 min. Following filtration through a 70 μm cell strainer, CD8⁺ T cells were purified by immunomagnetic separation using anti-CD8 microbeads. After isolation, CD8⁺ T cells were cultured with low dose IL2 and CD3/CD28 stimulatory beads to generate effector-like CD8+ T cells for in vitro assays.

Isolation of Human NK Cells and CD8+ T Cells from Peripheral Mononuclear Cells

Human NK cells and CD8+ T cells were isolated from blood of healthy, voluntary donors after they gave written and informed consent. Blood was mixed with PBS and loaded on Pancoll to isolate peripheral blood mononuclear cells (PBMCs) through centrifugation. NK cells were then isolated by negative selection using the Untouched Human NK Cell Isolation Kit (ThermoFisher). CD8+ T cells were then isolated by negative selection using the Untouched Human CD8+ T Cell Isolation Kit (Miltenyi).

Cell Treatment with PGE₂ or Tumor Factors

Murine or human NK cells or CD8⁺ T cells were treated with 1-100 ng/ml synthetic PGE₂ or medium as control for 16 h at 37° C. For experiments with tumor factors (TF), cells were incubated in a 1:1 mixture of complete RPMI medium with TF-containing conditioned medium from cancer cells. For the inhibitor studies, cells were first incubated with single EP receptor inhibitors (EP₁-EP₄) or inhibitor combinations at a concentration of 5 μM for 30 min, before adding 100 ng/ml PGE₂ for 16 h at 37° C. For the RNA and ATAC sequencing approach, NK cells were only pre-treated for 1 h with PGE₂ and then stimulated (as described below) for further 4 h.

### NK Cell Stimulation and Analysis of NK Cell Effector Function by Flow Cytometry

To assess cytokine and chemokine production and degranulation, NK cells were stimulated with plate-bound anti-NK1.1 (PK136), mimicking their activation in metastasis. Human NK cells were activated by incubation with the human lymphoma cell line K562. For analysis of cytokine and chemokine production, Brefeldin A was added 1 h after stimulation to block intracellular protein transport processes. 3 h later, cells were stained for flow cytometric analysis. For analysis of degranulation, stimulation was done in presence of anti-LAMP-1 antibody, and Monensin was added 1 h later to prevent acidification of endolytic vesicles.

For flow cytometric analysis, cells were resuspended in staining buffer containing antibodies for surface molecules and incubated for 15 min. A fixable viability dye was used to exclude dead cells in all experiments. Cells were fixed using the IC fixation buffer kit (Invitrogen) according to the manufacturer's instructions. Intracellular staining of cytokines (including IFNγ and TNF) was performed in permeabilization buffer and cells were subsequently analyzed by flow cytometry. Staining for intracellular Granzyme B was done using the Foxp3/Transcription factor buffer set from ebioscience.

CD8⁺ T Cell Stimulation and Analysis of CD8⁺ T Cell Function by Flow Cytometry

To assess T cell differentiation and expansion, CD8⁺ T cells were stimulated with anti-CD3/CD28 microbeads plus Interleukin-12 (10 ng/ml), mimicking their activation in metastasis or tumor tissue, and cell numbers were quantified 72 h later by flow cytometry.

### Cytokine Release Assay

To assess the production of cytokines in cell culture supernatants, NK cells were stimulated for 16 h and cell-free supernatants were harvested and stored at −20° C. until further use. Cytokine concentrations in the culture supernatants were measured by enzyme-linked immunosorbent assays (ELISA) or a multiplex cytokine assay (Bio-Plex Pro Mouse Cytokine 23-plex).

### NK Cell Cytotoxicity Assay

Measurement of the dynamics of NK cell cytotoxicity against BRAF^{V600E} tumor cells over time was performed with the impedance-based technology using an xCelligence RTCA MP device (ACEA Biosciences). The xCELLigence RTCA system measures changes in impedance generated by adherence of cells to electrodes on the bottom of the wells of E-Plates and calculates a parameter called Cell Index. For the assays, tumor cells were seeded in 96-well E-plates. After tumor cells reached confluency and a maximum cell index, in vitro cultured NK cells were added in different effector-to-target ratios. Killing was measured by declining cell index values. Percentage of specific cytotoxicity was finally calculated to the index values from tumor cells alone.

### RNA Isolation and Quantitative Real-Time PCR

RNA from NK cells was isolated using Monarch Total RNA Miniprep Kit and cDNA was generated using SensiFAST cDNA synthesis kit from Bioline. Primers against human XCL1/2 and HPRT were purchased from Eurofins. A mix of primer pairs, double-distilled water and 2×Takyon Mix SYBR green assay was added to the cDNA and amplified copies were quantified by LightCycler 480 (Roche).

### RNA-Sequencing

RNA was isolated from sorted or stimulated NK cell population using Monarch Total RNA Miniprep Kit. Following quality control by Agilent Bioanalyzer, RNA was subjected to cDNA synthesis. Barcoded cDNA of each sample was produced with a Maxima RT polymerase (Thermo Fisher) exercising oligo-dT primer containing barcodes, unique molecular identifiers (UMIs) and an adaptor. 5′ ends of the cDNAs were prolonged by a template switch oligo (TSO). After pooling all samples, full-length cDNA was amplified with primers binding to the TSO site and the adaptor. cDNA was supplemented with the Nextera XT kit (Illumina) and 3′-end-fragments finally amplified using P5 and P7 Illumina overhangs. The barcoded cDNA libraries were sequenced using a NextSeq 500 (Illumina).

### ATAC-Sequencing

ATAC Sequencing was performed as described previously (Buenrostro et al., Nature Methods, 2013). Briefly, NK cells were washed in cold PBS and lysed. The transposition reaction was incubated at 37° C. for 30 min. DNA was purified using the Monarch PCR & DNA Cleanup Kit and material was amplified for four cycles. After evaluation by real-time PCR, cycles were added as calculated. The amplified samples were purified and size selected using SpeedBeads. Libraries were controlled by BioAnalyzer and sequenced on a NovaSeq 6000 (Illumina).

### Bioinformatic Analysis of Cancer Patient Data

Gene expression datasets from The Cancer Genome Atlas (TCGA) were downloaded from Firehose (https://gdac. broadinstitute.org/). Gene signatures for functional NK cells contained a combination of NK cell-specific gene and genes identified to be suppressed by PGE2-signaling in our core PGE₂ gene expression dataset (NCR3, KLBR1, PRF1, CD160, NCR1, IFNγ, TNF, IL2, XCL1, CCL4, CCL15, CCL3, CCL1, CSF2, IL2Ra, CRTAM, ICAM1, TNFSF9, TNFSF14, TNFSF8). Overall survival analyses were performed for the top and bottom quartile ranked sum expression of signature genes and plotted for Kaplan-Meier curves using GraphPad Prism (GraphPad).

### Tumor Cell Injection for Metastasis Development

Tumor cells were harvested by trypsinization and washed three times in PBS. 1×10⁶ cells were intravenously injected in 100 μl endotoxin-free PBS to induce development of lung metastases.

### Tumor Cell Injection for Primary Tumor Growth

Tumor cells were harvested by trypsinization and washed three times in PBS. 1×10⁵ cells were subcutaneously injected into the flank of recipient mice to induce tumor development. Tumor growth was measured using a digital caliper. Tumor diameters stated in the figures refer to the average of the longest diameter and its perpendicular for each tumor.

### Analysis of Lung Metastases by Immunofluorescence Microscopy

Mice were anaesthetized in a chamber containing 45% isoflurane. Under deep anesthesia, mice were perfused with 10 ml PBS via the right ventricle of the heart to eliminate vascular blood content. In a second step, perfusion was continued with 10 ml Antigenfix solution (Diapath) to enable maximal epitope conservation. Pre-warmed 2% low melting point (LMP) agarose was gently injected into the trachea until lungs are inflated to full capacity. Organs were isolated and fixed for 4-6 h in Antigenfix. Following tissue fixation, lungs were embedded into melted 4% LMP agarose. Once solidified, agar tissue blocks were sectioned (300 μm thickness) using a VT1000S vibratome. Lung sections were permeabilized, blocked and stained in 0.1M Tris supplemented with 1% BSA, 0.3% Triton X-100 (Gerbu Biotechnik) and normal mouse serum. For staining actin filaments, tissue was incubated with Phalloidin for 2 h. Stained sections were mounted in Mowiol and analyzed on a confocal microscope with motorized stage (Zeiss) or a THUNDER imager (Zeiss). Image analysis was performed using Imaris software (Bitplane) on maximum projections of 11 Z-plane sections (20 μm thickness). Semi-automated analyses using the Imaris surface generation tool was used to reconstruct surfaces for mCherry⁺ metastases.

### Cytokine Neutralization In Vivo

For neutralization of IFNγ, 400 mg of anti-IFNγ antibody was injected intraperitonally (i.p.) two days after tumor cell injection. Antibody injections were performed every four days during the course of the experiment.

Adoptive Transfer of NK Cells into Rag2^{-/-}IL2rg^{-/-}

Conventional NK cells or RESURRECT NK cells (derived from GzmB^{Cre} Ptger2^{-/-}Ptger4^{fl/fl} mice) were isolated from spleen and cultured in low doses of IL2 and IL15/15Ra for five days. Meanwhile, BRAF^{V600E} mCherry tumor cells were intravenously injected into immunodeficient Rag2^{-/-}IL2rg^{-/-} mice. Three days after tumor cell injection, in vitro cultured NK cells were washed and adoptively transferred into mice. After further 11 days, metastatic livers were isolated and analyzed by immunofluorescence microscopy.

### Adoptive OT-I T Cell Transfer for Analysis of T Cell Therapy

For therapeutic T cell transfer, conventional OT-I CD8⁺ T cells were isolated from spleens of OT-I donor mice and RESURRECT OT-I CD8⁺ T cells were isolated from spleens of OT-I×CD4^{Cre} Ptger2^{-/-}Ptger4^{fl/fl} miceusing the naive CD8⁺ T cell isolation kit (Miltenyi Biotec). 1×10³ cells were adoptively transferred intravenously (i.v.) in sterile PBS into recipient WT mice previously transplanted s.c. with 2×10⁵ OVA-expressing BRAF^{V600E} melanoma cells.

The features disclosed in the foregoing description, in the claims and/or in the accompanying drawings may, both separately and in any combination thereof, be material for realizing the invention in diverse forms thereof.

### References

Bald, T., M. F. Krummel, M. J. Smyth, and K. C. Barry. 2020. The NK cell-cancer cycle: advances and new challenges in NK cell-based immunotherapies. Nature Immunology 21:1-13.
Böttcher J P, Bonavita E, Chakravarty P, Blees H, Cabeza-Cabrerizo M, Sammicheli S, Rogers N C, Sahai E, Zelenay S, Reis e Sousa C. NK Cells Stimulate Recruitment of cDC1 into the Tumor Microenvironment Promoting Cancer Immune Control. Cell. 2018; 172(5):1022-1037. e14. doi:10.1016/j.cell.2018.01.004.
Bonavita E, Bromley C P, Jonsson G, Pelly V S, Sahoo S, Walwyn-Brown K, Mensurado S, Moeini A, Flanagan E, Bell C R, Chiang S C, Chikkanna-Gowda C P, Rogers N, Silva-Santos B, Jaillon S, Mantovani A, Reis e Sousa C, Guerra N, Davis DM, Zelenay S. Antagonistic Inflammatory Phenotypes Dictate Tumor Fate and Response to Immune Checkpoint Blockade. Immunity. 2020; 53(6):1215-1229. e8. doi:10.1016/j.immuni.2020.10.020.
Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat. Methods 10, 1213-1218 (2013).
Burn, J., et al., Cancer prevention with aspirin in hereditary colorectal cancer (Lynch syndrome), 10-year follow-up and registry-based 20-year data in the CAPP2 study: a double-blind, randomised, placebo-controlled trial. Lancet, 2020; 395(10240): p. 1855-1863.
Glasner, A., A. Levi, J. Enk, B. Isaacson, S. Viukov, S. Orlanski, A. Scope, T. Neuman, C. D. Enk, J. H. Hanna, V. Sexl, S. Jonjić, B. Seliger, L. Zitvogel, and O. Mandelboim. 2018. NKp46 Receptor-Mediated Interferon-y Production by Natural Killer Cells Increases Fibronectin 1 to Alter Tumor Architecture and Control Metastasis. Immunity 48:107-119.e4.
Guillerey, C., N. D. Huntington, and M. J. Smyth. 2016. Targeting natural killer cells in cancer immunotherapy. Nat. Immunol. 17:1025-1036.
Guo, Q., et al., A comprehensive evaluation of clinical efficacy and safety of celecoxib in combination with chemotherapy in metastatic or postoperative recurrent gastric cancer patients: A preliminary, three-center, clinical trial study. Medicine (Baltimore), 2019; 98(27):p. e16234.
Lorenzo-Herrero, S., A. Lopez-Soto, C. Sordo-Bahamonde, A. Gonzalez-Rodriguez, M. Vitale, and S. Gonzalez. 2019. NK Cell-Based Immunotherapy in Cancer Metastasis. Cancers (Basel) 11:29-22.
Morvan, M. G., and L. L. Lanier. 2016. NK cells and cancer: you can teach innate cells new tricks. Nature Reviews Cancer 16:7-19.
Wang, D., and R. N. DuBois. 2016. The Role of Prostaglandin E2 in Tumor-Associated Immunosuppression. Trends Mol Med 22:1-3.
Zelenay, S., A. G. Van der Veen, J. P. Böttcher, K. J. Snelgrove, N. Rogers, S. E. Acton, P. Chakravarty, M. R. Girotti, R. Marais, S. A. Quezada, E. Sahai, and C. Reis e Sousa. 2015. Cyclooxygenase-Dependent Tumor Growth through Evasion of Immunity. Cell 162:1257-1270.

1. A modified natural killer (NK) cell or T cell or tumor-infiltrating lymphocyte (TIL), wherein the expression and/or activity of prostaglandin E receptor 2 (EP2) and of prostaglandin E receptor 4 (EP4) is selectively inhibited or eliminated.

2. The modified NK or T cell or TIL of claim 1, wherein the expression of prostaglandin E receptor 2 (EP2) and of prostaglandin E receptor 4 (EP4) is selectively inhibited or eliminated by genetic modification or wherein EP2 and EP4 are selectively inhibited or blocked by pharmacological inhibition.

3. The modified NK or T cell or TIL of claim 1,
which is genetically modified by knocking out EP2 and EP4, or
which is modified or edited by knocking out EP2 and EP4, or
which is modified by gene silencing/knockdown of EP2 and EP4.

4. The modified NK or T cell or TIL according to claim 1, which is further modified by introducing one or more components, selected from a chimeric antigen receptor (CAR), a chemokine or cytokine receptor, and an activating receptor; or is further modified by elimination of a gene encoding an inhibitory receptor.

7. (canceled)

8. A method for generating a modified NK or T cell or TIL according to claim 1, comprising the following steps:
(a) providing leukocytes, from a healthy subject/donor or from a patient,
(b) separating the NK or T cells or TILs and in vitro expanding the NK or T cells or TILs;
(c) genetically modifying the NK or T cells or TILs in order to selectively eliminate the expression of EP2 and EP4;
and
(d) in vitro expanding the modified NK or T cells or TILs.

9. The method of claim 8, wherein in step (a) the leukocytes, are
from periphery blood,
from cord blood,
from induced pluripotent stem cells, or
from tumor tissue (TILs),
and/or wherein the leukocytes are from autologous cells derived from a patient or are allogeneic cells derived from an unrelated healthy subject/donor.

10. The method of claim 8, wherein in step (c) the genetic modification is carried out by
using the CRISPR/Cas9 gene editing system, or
using zinc-finger nucleases (ZFNs), transcription activator-like effector nucleases (TALENs) and MegaTAL nucleases, or by using base editing, or
gene silencing/knockdown via introduction of short-hairpin RNAs via the Sleeping Beauty transposon system, lentiviral vectors or gamma-retroviral vectors.

11. The method according to claim 8, comprising an additional modification of the NK cell or T cell by introducing a component selected from a CAR, a chemokine or cytokine receptor, and an activating receptor; or is further modified by elimination of a gene encoding an inhibitory receptor.

12. A method for the treatment and/or prevention of cancer, comprising the step of
administering, to a subject in need thereof.
modified NK and/or T cells and/or TILs according to claim 1; or administering to the subject modified NK and/or T cells and/or TILs obtained by a method comprising the following steps:
(a) providing leukocytes from a healthy subject/donor or from a patient,
(b) separating the NK or T cells or TILs and in vitro expanding the NK or T cells or TILs;
(c) genetically modifying the NK or T cells or TILs in order to selectively eliminate the expression of EP2 and EP4; and
(d) in vitro expanding the modified NK or T cells or TILs.

13. The method of claim 12,
wherein, in step (a) the leukocytes are
from periphery blood,
from cord blood,
from induced pluripotent stem cells, or
from tumor tissue (TILs),
and/or wherein the leukocytes are from autologous cells derived from a patient or are allogeneic cells derived from an unrelated healthy subject/donor,
and/or wherein in step (c) the genetic modification is carried out by
using the CRISPR/Cas9 gene editing system, or
using zinc-finger nucleases (ZFNs), transcription activator-like effector nucleases (TALENs) and MegaTAL nucleases, or by using base editing, or
gene silencing/knockdown via introduction of short-hairpin RNAs via the Sleeping Beauty transposon system, lentiviral vectors or gamma-retroviral vectors.

14. A composition comprising:
(a) a modified NK and/or T cell and/or TIL of claim 1, and
(b) an excipient and/or carrier.

15. The composition of claim 14, wherein the modified NK or T cells or TILs are cryopreserved.

16. The method of claim 12, wherein the cancer is a solid tumor, a hematological tumor, and/or metastases, and the treatment and/or prevention of cancer comprises adoptive immune cell therapy.

17. The method of claim 16, wherein the adoptive immune cell therapy is selected from adoptive NK cell therapy, adoptive T cell therapy, CAR T cell therapy, CAR NK cell therapy and combinations thereof.

18. The method of claim 12, wherein the method comprises a combination therapy with one or more checkpoint blockade inhibitors targeting PD-1, CTLA-4, and/or PD-L1, and/or a combination with anti-programmed death (PD) protein 1 therapy.

19. The method of claim 19, wherein the leukocytes are lymphocytes.

20. The method of claim 8, wherein the leukocytes are lymphocytes.

21. The method of claim 8, wherein, in step (c), genetically modifying the NK or T cells or TILs in order to selectively eliminate the expression of EP2 and EP4 is done
by knocking out EP2 and EP4, or by gene silencing/knockdown of EP2 and EP4, or
modifying the NK or T cells or TILs by treating with EP2 and/or EP4 inhibitor(s).
